(19) United States
(12) Patent Application Publication
(10) Pub. No.: US 2024/0318267 A1
(43) Pub. Date: Sep. 26, 2024

(54) ONE-POT PATHOGEN DETECTION SYSTEM AND METHOD FOR REAL-TIME LATERAL FLOW ASSAY

(71) Applicant: City University of Hong Kong, Hong Kong (CN)

(72) Inventors: Mengsu YANG, Hong Kong (CN); Bereket AYELE, Hong Kong (CN); Renardi GUNAWAN, Hong Kong (CN); Wanqing WU, Hong Kong (CN)

(21) Appl. No.: 18/188,527

(22) Filed: Mar. 23, 2023

(51) Int. Cl. C12Q 1/70; G01N 33/543; G01N 33/569

(52) U.S. Cl. CPC C12Q 1/701; G01N 33/54388; G01N 33/56983; G01N 2333/065; G01N 2333/165

(57) ABSTRACT

The present disclosure provides a LAMP-based, one-pot pathogen detection system and method integrating a high specificity assay for identifying true positive samples in an analyte before a whole course of LAMP reaction completes, in order to shorten the reaction time and reduce the likelihood of false positive results due to non-specific amplification or signal error (e.g., sample has too high pH) in the LAMP reaction. The present invention integrates an immunochromatographic assay such as lateral flow assay (LFA) in result interpretation from colorimetric and/or fluorimetric aspects of the LAMP reaction in order to enable a real-time pathogen detection, thereby improving the efficiency thereof with high specificity. The present detection system employs a single reaction system to enable multiple result interpretation, and the LAMP reaction can be carried out in a simple platform capable of generating constant heat.

### REFERENCE TO SEQUENCE DISCLOSURE

The sequence listing file under the file name “P24729US00_sequence_listing.xml” submitted in ST.26 XML file format with a file size of 118 KB created on Mar. 14, 2023 is incorporated herein by reference.

### TECHNICAL FIELD

The present invention relates to a one-pot pathogen detection for real-time lateral flow assay system suitable for multiple detection modules in a single reaction.

### BACKGROUND

Since the COVID-19 pandemic occurred, the public health and medical system around the world have been challenged with ever-evolving new variants. Furthermore, with the recent monkeypox outbreak since July 2022 putting more strain in public health management, development of a simple and versatile point-of-care diagnostic test has been a focal point in pandemic control. Especially since the earlier disease and epidemics of the monkeypox virus (MPXV) happened in regions of central and West Africa where standard lab facilities and resources are not always available or very limited.

Loop-mediated isothermal amplification (LAMP) is a detection method developed by utilizing one enzyme for target detection in constant temperature with a possibility of real-time diagnosis by using one or more detection methods such as turbidimetry, colorimetry and fluorometry. The presence of amplification will cause changes in turbidity, color, or in fluorescence, thus allowing real-time result and could reduce the reaction time. However, these non-specific detection methods were reported to increase the likelihood of detecting false positive, even though LAMP usually utilizes 4-6 different primers to recognize 6-8 independent regions on a target sequence which in principle should result a higher specificity than that from a standard two-primer based PCR method. Some of these detection methods are also susceptible to external factors such as sample pH. Although compared with PCR, LAMP does not require cycling conditions and can be performed in simple heat block or water bath, simplifying its application in field use and in remote or poor regions.

On the other hand, utilizing lateral flow assay (LFA) to detect LAMP result gives a highly specific method to confirm the amplification result. LFA is an immunochromatographic method based on a prefabricated nitrocellulose strip containing antibodies to specific hapten, which the said haptens are incorporated in the target amplicons. The antigen-antibody reaction will capture the target amplicons containing the hapten, which are highly specific. (Posthuma-Trumpie et al., “Lateral flow (immuno)assay: its strengths, weaknesses, opportunities and threats. A literature survey”, Analytical and Bioanalytical Chemistry, 2009, 393, 569-582) Therefore, the problem with false positives can be mitigated by LFA-based interpretation. The drawback for this method is the lack of signal to indicate the amplification is done, thus all reaction must be performed at the end of a pre-defined maximum incubation duration.

A need therefore exists for a simple, versatile and reliable diagnostic system with minimal instrument requirements that is applicable in both standard lab setting and point-of-care testing, with fastest possible and minimal false-positive results. The proposed idea to integrate the real-time and LFA detection method for LAMP enables a detection of the result prior to maximum incubation time, depending on the concentration of target nucleic acid in the sample, with high specificity of the target. The combinations can mitigate the need to reach maximum incubation time, a standard in LFA-based LAMP such as described in patent WO2017103269A1 and CN101768641B, or false positive results in color-/fluorimetric-based “real-time” interpretation (Urrutia-Cabrera et al., “Comparative analysis of loop-mediated isothermal amplification (LAMP)-based assays for rapid detection of SARS-COV-2 genes”, Scientific Reports, 2021; 11: 22493)

### SUMMARY OF INVENTION

The present disclosure proposes a one-pot detection system utilizing the high specificity of LFA in combination with the real-time LAMP-based techniques such as colorimetric and fluorimetric in order to provide multiple readouts detectable by different diagnostic platforms. This is the first time to combine colorimetric, fluorimetric and LFA assays in a single reaction as they are generally performed independently and a mere combination of the afore-mentioned techniques risks a mutually suppressive reaction without any improvement in analytical performance.

Accordingly, a first aspect of the present invention provides a pathogen detection system including a reaction mixture for amplifying a target nucleic acid, where the reaction mixture includes:
a colorimetric indicator responsive to a change brought by a potential positive nucleic acid amplification in the reaction mixture;
a fluorescent indicator responsive to a presence of amplicons from an amplification reaction of each of target sequences;
a plurality of primers complementary to one or more independent regions in each of the target sequences to be amplified;
at least one enzyme for initiating the amplification reaction of the target sequences; and
a master mix for said amplification reaction,
the pathogen detection system further including a concurrent immunochromatographic assay, where the immunochromatographic assay includes an antibody specific to an identifier of the amplicons representing one of the target sequences.

Exemplarily, the amplification reaction of the target sequences is selected from loop-mediated isothermal amplification (LAMP) method.

In certain embodiments, some of the plurality of primers are conjugated with at least two different kinds of systematically selected tags such that the at least two kinds of tags are contained in corresponding amplicons as the identifier which is specifically bound with said antibody in the concurrent immunochromatographic assay.

In some embodiments, the at least two different kinds of tags conjugated with the plurality of primers are two different hapten tags.

In certain embodiments, the two different hapten tags are selected from any two of various hapten tags, including but not limited to biotin, 6-carboxyfluorescein (FAM), fluorescein isothiocyanate (FITC), digoxigenin (DIG), tetramethyl rhodamine (TAMRA), dinitrophenyl and sulforhodamine (Texas Red).

In certain embodiments, the two different hapten tags are conjugated to 5′-end of some of the plurality of primers except a forward outer primer (F3) and a reverse outer primer (B3).

In one embodiment, at least a reverse inner primer (BIP) or a forward inner primer (FIP) and a loop backward (LB) primer are conjugated with the two different hapten tags at their 5′-end, respectively.

In one embodiment, the BIP or FIP is conjugated with a biotin or FAM at its 5′-end and the LB primer is conjugated with a biotin or FAM at its 5′-end.

In other embodiments, a corresponding tag conjugated with some of the primers can be substituted with the other type of tag, and a corresponding antibody for specifically binding with the tag in the concurrent lateral flow assay is thereby substituted in order to validate the presence of the target sequence in the analyte.

In certain embodiments, the fluorescent indicator intercalates with the amplicons of at least one of the target sequences to emit fluorescence signal.

In certain embodiments, the fluorescent indicator is one or more fluorescent dyes selected from various intercalating dyes such as, but not limited to, SYBR green, SYTO-82, and SYTO-84.

In certain embodiments, the target sequence to be amplified into the amplicon with which said antibody specifically binds to the hapten-tag identifier thereof or the fluorescent indicator intercalates is a target sequence of a pathogen in the analyte.

In certain embodiments, the target sequences can consist of one or more target sequence of a pathogen, or one or more target sequence with one or more internal control sequences.

In certain embodiments, the pathogen from which the target sequence to be amplified in order for the antibody to specifically bind with or the fluorescent indicator to intercalate with includes, but not limited to, viruses, bacteria, fungi, and other types of pathogens.

In certain embodiments, the amplification of target nucleic acid drives changes in the reaction, including change in pH or ion concentrations such as Mg ions.

In certain embodiments, the colorimetric indicator is a colorimetric dye selected from various kinds of halochromic dyes or metallochromic dyes.

The halochromic dye includes, but not limited to, phenol red, methyl red, bromothymol blue, phenolphthalein, and triarylmethane; the metallochromic dye includes hydroxynaphtol blue.

In certain embodiments, the analyte includes, but not limited to, DNA molecules, RNA molecules, or combination of both DNA and RNA molecules.

In certain embodiments, the enzyme for initiating the amplification reaction of the target sequences includes DNA polymerase and reverse transcriptase, where the DNA polymerase may have reverse transcription capacity.

Preferably, the enzyme for initiating the amplification reaction of the target sequences has strand displacement activity.

The reverse transcriptase includes, but not limited to, Bst polymerase, AMV RT, M-MuLV RT. HIV RT. Superscript II RT, Thermoscript RT, and other possible RTs.

In certain embodiments, the target sequences include one or more target pathogen genes, and one or more host internal control genes.

The host internal control gene is a human internal control gene when the host of the target pathogen is human.

A second aspect of the present invention provides a method for improving detection efficiency and validity of a molecular diagnostic assay on a target pathogen gene in an analyte.

The method includes:
providing the reaction mixture of the pathogen detection system described herein;
initiating an amplification of target nucleic acid by enzymes in the reaction mixture in a platform capable of generating constant heat;
performing any colorimetric and/or fluorimetric analyses during said amplification by observing any change in color and/or spiked emission of fluorescence signal;
if any color change or spiked emission of fluorescence being observed, stopping said platform and subjecting reaction product obtained instantly after said stopping to an immunochromatographic assay; and
validating the result of said molecular diagnostic assay by a corresponding visible signal obtained from the immunochromatographic assay and comparing said visible signal with the color change or spiked emission of fluorescence; and
if said visible signal giving a coherent positive result representing a presence of the target pathogen gene in the analyte, a thermocycle at which said reaction product being validated as positive by said immunochromatographic assay representing an end of the molecular diagnostic assay.

In certain embodiments, the amplification of the target nucleic acid is performed at a constant reaction temperature.

In certain embodiments, the amplification of the target nucleic acid by enzymes in the reaction mixture directly includes loop-mediated isothermal amplification (LAMP) reaction.

The amplification of the target nucleic acid can also include other isothermal reactions without limiting to LAMP reaction.

In certain embodiments, said platform includes a thermocycler with fluorescence reading capability. In other embodiments, another platform such as heat block or water bath can be utilized for the reaction.

In certain embodiments, the amplification of the target nucleic acid by enzymes in the reaction mixture directly is performed constantly at a specific temperature in a specific time frame. For example, said reacting can be performed at a temperature ranging from 63° C. to 65° C.

The amplification of the target nucleic acid can last as low as 10 minutes to 45 minutes, depending on the primer set selected.

In certain embodiments, the color change is due to a halochromic change or metallochromic change during said reacting the reaction mixture directly with the analyte, and indicative of a presence of amplicons from said reaction

In certain embodiments, the spike of fluorescence signal emission is indicative of the presence of amplicon(s) from the amplification reaction of the reaction mixture directly with the analyte.

In certain embodiments, the fluorescence signal comes from one or more fluorescent dyes capable of intercalating the amplicon from the amplification reaction of the reaction mixture directly with the analyte.

The one or more fluorescent dyes include, but not limited to, SYBR green, SYTO-82, and SYTO-84.

In certain embodiments, the colorimetric, fluorimetric and immunochromatographic assays are performed in an integrated device.

Preferably, the integrated device for performing the colorimetric, fluorimetric and immunochromatographic assays includes a colorimetric and fluorimetric detector for monitoring any color change and/or spike emission of fluorescence signal in real-time during said reaction.

In other embodiments, the immunochromatographic assay is performed in a separate lateral flow device or integrated with the main reaction.

Preferably, the immunochromatographic assay is selected from a lateral flow assay (LFA).

Other aspects of the present invention include providing a kit for use in a one-pot pathogen detection in an analyte including the pathogen detection system described herein. Different indicators and amplification reaction components of the pathogen detection system described herein can be formulated in wet or dry chemistry form.

Preferably, the amplification reaction components including the reaction mixture, primers, enzyme(s), dNTPs, and buffers for amplification reaction and the indicators, are formulated in dry chemistry form.

In certain embodiments, the amplification reaction components and one or more indicators are formulated in a plurality of dry reagents. The dry beads will be re-constituted into water before use.

In other embodiments, extraction-free buffer may be used to re-constitute the dry beads in direct extraction testing.

In other embodiments, the reaction mixture and indicators of the pathogen detection system can be formulated in wet chemistry form.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The patent or application file contains at least one drawing executed in color. Copies of this patent or patent application publication with color drawing(s) will be provided by the Office upon request and payment of the necessary fee.

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1 shows a result of sequence alignment between various MPXV and non-MPXV poxvirus sequences listed in Table 2 obtained from online databases containing TNF-α receptor encoding sequence (Table 3) for prediction of TNF LAMP primer sequences (Table 1);

FIG. 2 shows a result of sequence alignment between various MPXV and non-MPXV poxvirus sequences listed in Table 2 obtained from online databases containing encoding sequence of A-type inclusion (ATI) bodies (Table 3) for prediction of ATI LAMP primer sequences (Table 1);

FIG. 3 displays a gel electrophoresis image for initial MPXV primer set and temperature screening. Each primer set was tested with positive sample (+) and NTC in 59° C. to 65° C.

FIG. 4 shows the comparison of P2 and P10 primer sets of MPXV TNF primers tested at (left) 65° C. and (right) 63° C. in 2 different sample concentrations (1:100 k and 1:1M dilutions).
* denotes P value<0.01;

FIG. 5 illustrates the effect of loop primer LF and LF addition into P2 basic primer set.
* denotes P value<0.05;

FIG. 6 shows an image of an agarose gel for gel electrophoresis assay of TNF LAMP primer set optimization using positive control sample of MPXV in various sample concentrations;

FIG. 7 shows Cq values of multiple primer tagging combination methods (Methods 6 to 11, or hereinafter as “M6, M7, M8, M9, M10, and M11”, respectively) of MPXV TNF primer set (left). The primer tagging methods with lowest Cq, thus most efficient reaction, was compared with untagged primer set (M1); n.s. denotes the non-significant difference (p value >0.05);

FIG. 8 displays images of LFA result from the multiple primer tagging test in FIG. 7; (left) the best 3 Methods with the lowest Cq values, M6, M9 and M10, were tested for positive result, displaying all positive in the samples; (right) the NTCs of all Methods were screened in LFA for false positive results.

FIG. 9 shows (a) a comparison of standard (non-hapten-tagged) TNF LAMP primers with hapten-tagged LAMP primers in terms of gel electrophoresis after amplification using positive control sample of MPXV in different dilutions; NTC is a negative control and (b) a result of LFA of the positive samples obtained from the hapten-tagged primers according to FIG. 9a;

FIG. 10 shows (a) gel electrophoresis picture and (b) LFA results of the optimized MPXV TNF primer set in various sample concentrations. The same sample was run on gel and LFA in the same sequence;

FIG. 11 illustrates the limit of detection (LoD) calculation for optimized primer set using Probit curve; Cq values from FIG. 10 were used to construct Probit curve to determine the sample concentration where the reaction has 95% detection rate;

FIG. 12 shows the specificity test of the TNF primer set on several bacterial DNA samples (1-5), other virus (SARS-COV-2 N gene, 6), human DNA (HeLa cell line, 7), and several non-MPXV orthopoxvirus sequences (8-13). LFA tests on bacterial DNA samples are included;

FIGS. 13A-13D show a multiple result interpretation of the one-pot detection system according to certain embodiments, in which: FIG. 13A shows fluorimetric change (intercalating fluorescence from DNA amplicon of a target sequence); FIG. 13B shows colorimetric change (pH change); FIG. 13C shows results of LFA; and FIG. 13D shows the result of gel electrophoresis;

FIG. 14A illustrates a fluorimetric assay result of ten control samples of MPXV (in the table: samples 1-7: positive control with MPXV in different dilutions (from 1:100 k to 1:5M fold); samples 8-10: negative control (“NTC”)) prepared according to certain embodiments of the present invention;

FIG. 14B shows images of a colorimetric assay result of the ten control samples prepared according to the table in FIG. 14A;

FIG. 14C shows an image of a gel electrophoresis result of the ten control samples prepared according to the table in FIG. 14A;

FIG. 14D shows a LFA result of the ten control samples prepared according to the table in FIG. 14A;

FIG. 15 shows a comparison between the data obtained from a full 45-minute amplification reaction (end point test) and the data obtained from the amplification reaction stopped right after the fluorescence reading spikes up in 18 minutes (real time test) in terms of LAMP Cq value and LFA result of a positive and negative control samples of MPXV prepared by the present detection system according to certain embodiments;

FIG. 16A shows images of colorimetric assay results of a positive control (in 1:10 k and 1:100 k dilutions) and a no-template control (“NTC”, or interchangeably named as negative control) of MPXV recorded at initial time point (left image), after a full 45-minute LAMP reaction (top row in right image) and when the colorimetric dye color is changed from red to yellow (bottom row in right image) according to certain embodiments of the present invention;

FIG. 16B shows images of colorimetric assay results for the color change in positive control samples of MPXV at different dilutions incubated with the LAMP reaction components for 12 and 15 minutes, respectively, and their corresponding LFA result recorded real time (12 or 15 minutes) and at the end point (˜45 minutes) according to certain embodiments of the present invention;

FIG. 17 shows a gel electrophoresis result (image of an agarose gel) of ATI MPXV primer set optimization by reacting with various concentrations of positive control of MPXV from 10 k dilution up to 1 billion (1 B) times dilution;

FIGS. 18A-18C show the result of hapten-tagged ATI LAMP primer compared with untagged primer set, in which: FIG. 18A shows the result of gel electrophoresis; FIG. 18B shows that the Cq value of the samples in the gel was compared to untagged primers, and statistical test shows that the hapten tagging has no effect on the primers efficiency; FIG. 18C shows that LFA result of the tagged primer samples is in line with the gel result as in FIG. 18A;

FIG. 19 shows the test on ATI LAMP primer with colorimetric reaction and LFA reading module with 1:100 k diluted positive control of MPXV;

FIG. 20 displays the test on ATI LAMP primer with colorimetric reaction and LFA reading module with 1M diluted positive control of MPXV;

FIG. 21 shows an alignment of SARS-COV-2 E gene in various known variants;

FIG. 22 shows the screening result of 3 primer sets E1, E2 and E3. (Top) Comparison of 3 primer sets ran on 4 temperatures. Statistical tests were performed to compare 63° C. result with next 2 best temperatures. E2 primer set were further analyzed (insert) (Bottom) Comparison of amplification efficiency between 3 primer sets in all temperatures. *** denotes the p value <0.001; ** denotes the p value <0.01; * denotes the p value <0.05; n.s. denotes the non-significant difference (p value >0.05);

FIG. 23 displays gel electrophoresis result of temperature screening for 3 primer sets from FIG. 22 test. The primers were run in 4 different temperature, with 3 replicates of each primer sets (marked black numbers) and NTC (marked in white numbers);

FIG. 24 shows Cq values of multiple primer tagging combination methods (Methods 6 to 11, or hereinafter as “M6, M7, M8, M9, M10, and M11”, respectively) and statistical test on each Method for targeting Covid-19 envelope (E) gene in a real-time LFA-based LAMP assay according to certain embodiments; *** denotes the p value <0.001; n.s. denotes the non-significant difference (p value >0.05);

FIG. 25 shows the LFA analysis result of the top three primer tagging combination methods (M6-M8) according to FIG. 23: tests on M6-M8 with 1000 copies (cps) per sample (left) give positive result in all methods, whereas tests on NTCs of M6-M8 show a false positive in M7 method (right);

FIG. 26 shows the (left) Cq values in 1000 and 200 copies/reaction samples between M6 and M8 according to FIGS. 23 and 24, where statistical analysis shows no difference between the two methods; (right) the effect of hapten tagging on the primers according to FIGS. 23-25, in which M8 primer set performance was compared to that of untagged primer set (M1); statistical analysis thereof shows no significant difference between the tagged and untagged primer sets;

FIG. 27 shows gel electrophoresis results from specificity test of E2 primer set with human DNA extracted from Human DNA (HeLa and 293T cell lines) (Top), and bacterial DNA (middle). NTC: non-template control;

FIG. 28 displays the result of real-time LFA-based LAMP using E2 primer set. The color changes of the reaction are displayed along with the LFA result. Samples 1˜4 used 20000 copies/reaction, while sample 5-8 used 200 copies/reaction. The samples were stopped in different times, as written in Table 24;

FIG. 29 shows a LFA analysis in the real-time LFA-based LAMP assay on 500 copies/reaction sample incubated in three different time intervals (5, 10, and 15 mins) and compared to NTC: the color of the samples progressively becomes more orange with a longer incubation. The change of color into slightly orange in the 10-minute incubation sample relative to NTC shows that the amplification is underway;

FIG. 30 shows the fluorescence graph of samples obtained at different time intervals according to FIG. 29 in the real-time LFA-based LAMP assay: arrows denote the time when samples were removed (5 and 10 mins) or the end of reaction (15 mins). The graph peaks at 10^{th} and 15^{th} minute are both above the threshold line, signifying the positive result for both cases;

FIG. 31 shows the result of the LFA analysis for each sample obtained at 5, 10, and 15 minutes from the real-time LFA-based LAMP reaction, and NTC. LFA result is in consensus with both color and fluorescence result shown in FIGS. 29 and 30, respectively.

Skilled artisans will appreciate that elements in the figures are illustrated for simplicity and clarity and have not necessarily been depicted to scale.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

The present disclosure provides systems and methods, as well as the components required to enable the systems and methods. Molecular diagnostics using the LAMP system has been used for more than a decade and become popular during the COVID-19 outbreak since 2020, becoming a viable alternative to gold standard qPCR, which despite its sensitivity and reliability, requires advanced equipment, controlled laboratory settings, well-trained laboratory personnel and lengthy procedures. The relative simplicity and versatility and more robust reaction with higher tolerance to inhibitors make LAMP a potential point-of-care technology (POCT), yet there are still problems plaguing LAMP adaptation as standard molecular diagnostic. The widespread use of LAMP as a conventional rapid diagnostic tool appears to be still challenging due to occasional reports of false positives in the conventional result interpretations.

Also, the lack of specificity in LAMP result interpretation is compounded with limited real-time measurement method. An end-point result interpretation, such as gel electrophoresis of restriction enzyme digest or using LFA-based reading, gives more specific result in return of requiring full reaction time, while LAMP result interpretation methods with real-time capacity, for example, colorimetric or fluorimetric readings, are prone to readings error or false positives. To address individual drawback of each of the result interpretation methods, the present invention incorporates a highly specific and easy-to-use LFA system alongside the real-time readings by colorimetric and/or fluorimetric methods into one single reaction. Users of the present system may only need to add a test sample or analyte in high temperature briefly, then directly into the reaction mixture or incubate briefly in high temperature, depending on the sample type, followed by incubation at the required reaction temperature for a certain period of time until a color or fluorimetric change indicating the presence of reaction product is observed, then the reaction can be stopped accompanied by an automatic or manual immunochromatographic assay such as lateral flow assay (LFA) to provide a highly specific readout through identification of two distinct tags on the amplicon by antibodies in LFA strip. To enable an identifier to be recognized by the antibody during the LFA, some LAMP primers are systematically selected for modification by two distinct tags. In the following examples and some embodiments of the present invention, biotin and FAM are used to conjugate to the 5′-end of FIP or BIP and LB primers, respectively, in a set of LAMP primers for amplifying the target sequence. It should be understood that these tags can be conjugated to 5′-end of other LAMP primers except F3 and B3 primers. A thorough screening method is performed to screen the best tag combination. The corresponding primer sequences for a set of LAMP primers to target MPXV-containing samples are provided in Example 1 and Table 1 described hereinafter. Examples of screening method for the tagged LAMP primers for LFA is provided in Examples section.

Preferably, these tags are haptens conjugated with one or more of, but not limited to, biotin, fluorescein such as FITC and FAM, and/or digoxigenin (DIG). In certain embodiments, 5′-end of BIP primer is tagged with biotin while 5′-end of LB primer is tagged with FAM. In other embodiment, biotin is tagged into 5′-end of FIP primer and FAM is tagged to 5′-end of LB primer.

The term “one-pot detection” or alike described herein refers to a combination of different detection and result interpretation platforms including, but not limited to, colorimetric assay, fluorimetric assay, immunochromatographic assay or more specifically, lateral flow assay when the immunochromatographic assay is carried out on a lateral flow device, and gel electrophoresis of LAMP reaction products. These assays will involve a combination of different indicators including, but not limited to, halochromic dye (e.g., phenol red, methyl red, bromothymol blue, phenolphthalein, triarylmethane, etc.), fluorescence dye (e.g., SYBR green, SYTO-82, SYTO-84, etc.), antibody for identifying tags on the amplicon of the target sequence, and LAMP reaction mixture including primers for amplifying the target sequence. Alternative to the halochromic dye, metallochromic dye such as hydroxynaphtol blue (HNB) which responses to Mg²⁺ ions generated during the LAMP reaction can be used as an indicator of the colorimetric assay as it is unaffected by the pH of the test samples.

The following examples are intended to assist the illustration of various embodiments of the present invention. Scope of invention should be referred to the appended claims.

### EXAMPLE 1

MPXV-specific TNF LAMP primers were designed using LAMP primer design software (such as PrimerExplorer) with certain modifications, except SEQ ID NOS: 5 and 6 which were designed by the present inventors due to the limitation of the software. In certain additional examples described hereinafter, another ATI LAMP primer set will be used in a concurrent LFA test. Before the ATI primer sets were used in the said examples, the primer sets were evaluated and modified accordingly.

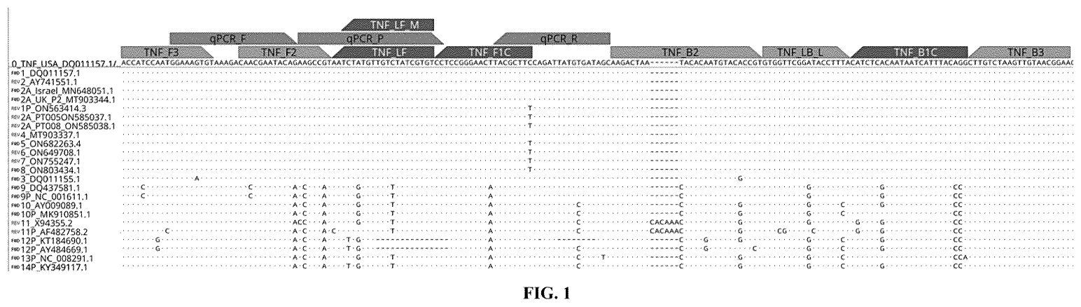

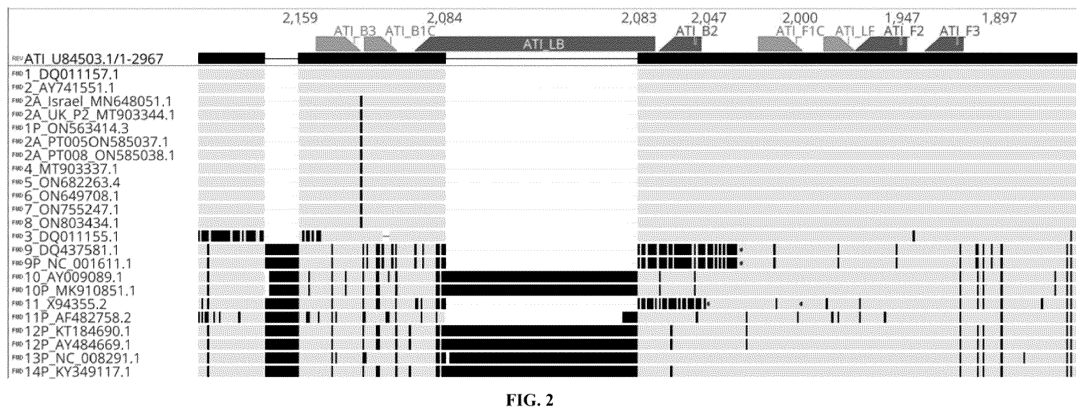

FIGS. 1 and 2 shows a sequence alignment of various MPXV and non-MPXV poxvirus sequences obtained from NCBI database to predict a set of LAMP primer sequences specific to TNF-alpha receptor (also known as cytokine response modifier (Crm) B) in poxviruses and an a-type inclusion (ATI) bodies, another biomarker of MPXV being formed in cytoplasm of host cells infected by MPXV, which are filled with mature MPXV virions. These sequences were selected to represent the pre and post-2022 outbreak of MPXV. FIGS. 1 and 2 displayed the in-silico specificity analysis of TNF and ATI LAMP primers, respectively, to MPXV consensus sequences to avoid potential false positive reaction arising from closely related non-MPXV poxviruses, if any, in the same analyte. Table 1 lists out the final sequences of various TNF and ATI LAMP primer sets for MPXV and modifications made to the primer sequences. In certain additional examples described hereinafter, these TNF and ATI LAMP primers will be used in a concurrent LFA test. Before the primer sets were used in said examples, the primer sets were evaluated and modified accordingly.

The positive control samples used in certain examples of the present disclosure for the detection of the presence of MPXV related targets contain an encoding gene for TNF-alpha receptor and a-type inclusion (ATI) bodies. Various partial sequences relating to the TNF-α receptor gene of the poxviruses were obtained from NCBI (Table 2) to design the LAMP primers specific to the TNF and ATI regions of MPXV. For example, an extracted sequence from 195878-196392 bp of the Monkeypox virus strain USA_2003_039, complete genome (with an accession ID of DQ011157.1 to GenBank of NCBI) (SEQ ID No: 50) is used as a reference to align with the corresponding regions of genome of other monkeypox viruses and orthopoxviruses as set forth in Table 2 for designing different TNF LAMP primers and the positive control plasmid (SEQ ID No: 22). Analogously, an extracted sequence from 1916 to 2150 bp of Monkeypox virus A-type inclusion body protein gene, complete cds (with an accession ID of U84503.1) (SEQ ID No: 73) is used as a reference to align with the corresponding regions of genome of other monkeypox viruses and orthopoxviruses as set forth in Table 2 for designing positive control plasmid for ATI primer set (SEQ ID No: 21).

TABLE 1

| SEQ | | | | |
|---|---|---|---|---|
| ID | MPXV | Primer | | |
| NO: | target | name | Sequence | Modifications |
| 1 | TNF | FIP_TNF | 5′- | N/A |
| | | | GAAGCGTAAGTTCCCGGAGGCAACGAATA | |
| | | | CAGAAGCCGT-3′ | |
| 2 | TNF | F3_TNF | 5′-ACCATCCAATGGAAAGTGT-3′ | N/A |
| 3 | TNF | BIP_TNF | 5′- | N/A |
| | | | AAGACTAATACACAATGTACACCGTCCTGT | |
| | | | AAATGATTATTGTGAGATG-3′ | |
| 4 | TNF | B3_TNF | 5′-TTCCGTTACAACTTAGACAAG-3′ | N/A |
| 5 | TNF | LF_TNF | 5′-ACACGATAGACAACATAGATT-3′ | N/A |
| 6 | TNF | LB_TNF | 5′-GTGGTTCGGATACCTTTA-3′ | N/A |
| 7 | TNF | FIP_BIO_ | 5′- | 5′-Biotin |
| | | TNF | GAAGCGTAAGTTCCCGGAGGCAACGAATAC | labeled |
| | | | AGAAGCCGT-3′ | |
| 8 | TNF | BIP_BIO_ | 5′- | 5′-Biotin |
| | | TNF | AAGACTAATACACAATGTACACCGTCCTGT | labeled |
| | | | AAATGATTATTGTGAGATG-3′ | |
| 9 | TNF | LB_BIO_ | 5′-GTGGTTCGGATACCTTTA-3′ | 5′-Biotin |
| | | TNF | | labeled |
| 10 | TNF | FIP_FAM_ | 5′- | 5′-FAM |
| | | TNF | GAAGCGTAAGTTCCCGGAGGCAACGAATAC | labeled |
| | | | AGAAGCCGT-3′ | |
| 11 | TNF | LF_FAM_ | 5′-ACACGATAGACAACATAGATT-3′ | 5′-FAM |
| | | TNF | | labeled |
| 12 | TNF | LB_FAM_ | 5′-GTGGTTCGGATACCTTTA-3′ | 5′-FAM |
| | | TNF | | labeled |
| 13 | ATI | FIP_ATI | 5′- | N/A |
| | | | TGGAGTCTGCTAATCTCTGTAAGATTAGAGA | |
| | | | ACTAGAGAATAAGTTGACC-3′ | |
| 14 | ATI | F3_ATI | 5′-CACAAGAAGTTGATGCACTG-3′ | N/A |
| 15 | ATI | BIP_ATI | 5′- | N/A |
| | | | TGAGTGAATGCCGTGGAAATGCGCAGTCGTT | |
| | | | CAACTGTA-3′ | |
| 16 | ATI | B3_ATI | 5′-CAGCATTGATTTCATTATTACGT-3′ | N/A |
| 17 | ATI | LF_ATI | 5′-CGCTCTCGATGCAGTC-3′ | N/A |
| 18 | ATI | LB_ ATI | 5′-CAGAGATTACAATCTAGAATCTCAG-3′ | N/A |
| 19 | ATI | BIP_BIO_ | 5′- | 5′-biotin |
| | | ATI | TGAGTGAATGCCGTGGAAATGCGCAGTCGTT | labeled |
| | | | CAACTGTA-3′ | |
| 20 | ATI | LB_FAM_ | 5′-CAGAGATTACAATCTAGAATCTCAG-3′ | 5′-FAM |
| | | ATI | | labeled |

TABLE 2

| Database name | Accession ID | Remarks |
|---|---|---|
| Monkeypox virus A-type inclusion body | U84503.1 | Reference for ATI gene |
| protein gene, complete cds | | alignment (SEQ ID No.: 73) |
| Monkeypox virus strain USA_2003_039, | DQ011157.1 | Reference for TNF gene |
| complete genome | | alignment (SEQ ID No: 50); |
| | | ATI region for alignment: |
| | | SEQ ID No: 74 |
| Monkeypox virus isolate Sierra Leone, | AY741551.1 | Pre-2022 outbreak MPXV |
| complete genome. | | sequences (TNF region: SEQ |
| | | ID No: 51; ATI region: SEQ |
| | | ID No: 75) |
| Monkeypox virus strain Israel_2018, | MN648051.1 | Pre-2022 outbreak MPXV |
| complete genome | | sequences (TNF region: SEQ |
| | | ID No: 52; ATI region: SEQ |
| | | ID No: 76) |
| Monkeypox virus isolate MPXV-UK_P2, | MT903344.1 | Pre-2022 outbreak MPXV |
| complete genome | | sequences (TNF region: SEQ |
| | | ID No: 53; ATI region: SEQ |
| | | ID No: 77) |
| Monkeypox virus isolate | ON563414.3 | Reference sequence from |
| MPXV_USA_2022_MA001, complete | | 2022 MPXV outbreak (TNF |
| genome | | region: SEQ ID No: 54 ATI |
| | | region: SEQ ID No: 78) |
| Monkeypox virus isolate | ON585037.1 | Sequence from 2022 MPXV |
| Monkeypox/PT0005/2022, complete | | outbreak (TNF region: SEQ |
| genome | | ID No: 55; ATI region: SEQ |
| | | ID No: 79) |
| Monkeypox virus isolate | ON585038.1 | Sequence from 2022 MPXV |
| Monkeypox/PT0008/2022, complete | | outbreak (TNF region: SEQ ID |
| genome | | No: 56; ATI region: SEQ ID |
| | | No: 80) |
| Monkeypox virus isolate MPXV- | MT903337.1 | Sequence from 2022 MPXV |
| M2940_FCT, complete genome | | outbreak (TNF region: SEQ ID |
| | | No: 57; ATI region: SEQ ID |
| | | No: 81) |
| Monkeypox virus isolate | ON682263.4 | Sequence from 2022 MPXV |
| MPXV/Germany/2022/RKI03, complete | | outbreak (TNF region: SEQ ID |
| genome | | No: 58; ATI region: SEQ ID |
| | | No: 82) |
| Monkeypox virus isolate | ON649708.1 | Sequence from 2022 MPXV |
| Monkeypox/PT0023/2022, partial | | outbreak (TNF region: SEQ ID |
| genome | | No: 59; ATI region: SEQ ID |
| | | No: 83) |
| Monkeypox virus isolate | ON755247.1 | Sequence from 2022 MPXV |
| MPXV/Germany/2022/RKI041 | | outbreak (TNF region: SEQ ID |
| | | No: 60; ATI region: SEQ ID |
| | | No: 84) |
| Monkeypox virus isolate | ON803434.1 | Sequence from 2022 MPXV |
| Mpx V/human/CAN/UN-NML- | | outbreak (TNF region: SEQ ID |
| 2886/2022, partial genome | | No: 61; ATI region: SEQ ID |
| | | No: 85) |
| Monkeypox virus strain Zaire_1979-005, | DQ011155.1 | Sequence from 2022 MPXV |
| complete genome | | outbreak (TNF region: SEQ ID |
| | | No: 62; ATI region: SEQ ID |
| | | No: 86) |
| Variola virus strain Bangladesh 1975 | DQ437581.1 | Variola virus (TNF region: SEQ |
| v75-550 Banu, complete genome | | ID No: 63; ATI region: SEQ ID |
| | | No: 87) |
| Variola virus, complete genome | NC_001611.1 | Variola virus; used as negative |
| | | control (TNF region: SEQ ID |
| | | No: 64; ATI region: SEQ ID |
| | | No: 88) |
| Camelpox virus CMS, complete genome | AY009089.1 | Camelpox virus (TNF region: |
| | | SEQ ID No: 65; ATI region: |
| | | SEQ ID No: 89) |
| Camelpox virus strain Negev2016, | MK910851.1 | Camelpox virus; used as |
| complete genome | | negative control (TNF region: |
| | | SEQ ID No: 66; ATI region: |
| | | SEQ ID No: 90) |
| Cowpox virus strain GRI-90, complete | X94355.2 | Cowpox virus (TNF region: |
| genome | | SEQ ID No: 67; ATI region: |
| | | SEQ ID No: 91) |
| Cowpox virus strain Brighton Red, | AF482758.2 | Cowpox virus; used as negative |
| complete genome | | control (TNF region: SEQ ID |
| | | No: 68; ATI region: SEQ ID |
| | | No: 92) |
| Vaccinia virus strain IOC clone B141, | KT184690.1 | Vaccinia virus; used as negative |
| complete genome | | control (TNF region: SEQ ID |
| | | No: 69; ATI region: SEQ ID |
| | | No: 93) |
| Rabbitpox virus, complete genome | AY484669.1 | Rabbitpox virus; used as |
| | | negative control (TNF region: |
| | | SEQ ID No: 70; ATI region: |
| | | SEQ ID No: 94) |
| Taterapox virus, complete genome | NC_008291.1 | Taterapox virus; used as |
| | | negative control (TNF region: |
| | | SEQ ID No: 71; ATI region: |
| | | SEQ ID No: 95) |
| Horsepox virus strain MNR clone 2-1, | KY349117.1 | Horsepox virus; used as |
| complete genome | | negative control (TNF region: |
| | | SEQ ID No: 72; ATI region: |
| | | SEQ ID No: 96) |

All finalized LAMP primer sequences were sent to Synbio Technologies for production. Based on the alignment results of FIGS. 1 and 2, consensus TNF and ATI genes of MPXV (Table 3) were selected and produced in plasmid format by Synbio Technologies as well. Preferably, the positive control sample contains multiple target sequences for present disclosure, including consensus ATI and TNF sequences. Preferably, the plasmid form was produced in dry form at 5 mg to be reconstituted in 1 ml of RNAse-free water as a stock. This stock could be diluted into up to a million times as positive control samples of MPXV for use in various examples and embodiments of the present disclosure. The number of plasmids in different dilution samples can be quantified by qPCR using the primers and probe provided in Table 4 and following the PCR amplification components and reaction conditions set forth in Tables 5 and 6, respectively.

TABLE 3

| SEQ ID | Species | Target | |
|---|---|---|---|
| NO: | origin | region | Sequence |
| 21 | MPXV | ATI | GACACACAAGAAGTTGATGCACTGCGTTCG |
| | | | CGTATTAGAGAACTAGAGAATAAGTTGAC |
| | | | CGACTGCATCGAGAGCGGAGGAGGAAATC |
| | | | TTACAGAGATTAGCAGACTCCAATCTAGAA |
| | | | TCTCAGATCTTGAAAGACAACTGAGTGAAT |
| | | | GCCGTGGAAATGCTACAGAGATTACAATCT |
| | | | AGAATCTCAGATCTTGAAATACAGTTGAAC |
| | | | GACTGCGGACGTAATAATGAAATCAATGCT |
| | | | GATATGGAAAAGAGATAGAATCATGGATC |
| | | | TTGATAGACATCTTAACGATTGTAAAAACG |
| | | | GTAACGGAGCATCTTCTGAGGAGGTAAAT |
| | | | AGGCTAAAGACTAGAATCAGGGATCTTGA |
| | | | ACGATCGCTAGAGATCTTCTCAAAGGATGA |
| | | | ATCAGAACTCTATTCGGCATATAAAACTGA |
| | | | ACTCGGTAATATCAGCACTTAATAATAAAC |
| | | | GGAAGAGATATAGCACCACATGCACCATC |
| | | | CAATGGAAAGTGTAAAGACAACGAATACA |
| | | | GAAGCCGTAATCTATGTTGTCTATCGTGTC |
| | | | CTCCGGGAACTTACGCTTCCAGATTATGTG |
| | | | ATAGCAAGACTAATACACAATGTACACCGT |
| | | | GTGGTTCGGATACCTTTACATCTCACAATA |
| | | | ATCATTTACAGGCTTGTCTAAGTTGTAACG |
| | | | GAAGATG |
| 22 | MPXV | TNF | TTAAGCCGCTAGAAGTTTTCCGTTTGATAT |
| | | | AGGATGTGGACATTTAACAATCTGACACGT |
| | | | GGGTGGATTGGACCATTCTCCTCCTGAACA |
| | | | CATGACACCAGAGTTACCAATCAACGAAT |
| | | | ATCCACTATTGCAACTATAAGTTACAATGC |
| | | | TCCCATCGATATAAAAATCCTCGTATCCGT |
| | | | TATGTCTTCCGTTGGATATAGATGGAGGTG |
| | | | ATTGGCATTTAACAGATTCGCAAATAGGTG |
| | | | CCTCAGGATTCCATACCATAGATCCAGTAG |
| | | | ATCCTAATTCACAATACGATTTAGATTCAC |
| | | | CGATCAAATGATATCCGCTATTACAAGAGT |
| | | | ACGTTATACTAGAGCCAAAGTCTACTCCGC |
| | | | CAATATCAAGTTGGCCATTATCGATATCTC |
| | | | GAGGCGATGGGCATCTCCGTTTAATACATT |
| | | | GATTAAAGAGTGTCCATCCGGTACCGGTAC |
| | | | ATTTAGCATATATGGGTCCCATTTTTTGCTT |
| | | | TCTGTATCCAGGTAGACATAGATATTCTAT |
| | | | AGTGTCTCC |

TABLE 4

| SEQ | | | | |
|---|---|---|---|---|
| ID | MPXV | Primer | | |
| NO: | target | name | Sequence | Modifications |
| 23 | ATI | PCR_F | 5′-GGAAAATGTAAAGACAACGAATACAG- | N/A |
| | | | 3′ | |
| 24 | ATI | PCR_R | 5′-GCTATCACATAATCTGGAAGCGTA-3′ | N/A |
| 25 | ATI | PCR_P | 5′FAM- | 5′-FAM and |
| | | | AAGCCGTAATCTATGTTGTCTATCGTGTCC- | 3′-BHQ |
| | | | 3′BHQ1 | quencher |

TABLE 5
PCR amplification components

| | | | | |
|---|---|---|---|---|
| | | Supplier/ | | Final |
| | | Manu- | Mixed | Concen- |
| No. | Reagents | facturer | volume | tration |

PCR components

| | | | | |
|---|---|---|---|---|
| 1 | Mastermix | Quantabio | 5 μL (25% | 1x |
| | (Ultraplex ™ 1- | | total volume) | |
| | step Toughmix ® 4X) | | | |
| 2 | Primer - probe mix | Synbio | 2 μL (10% | 1x |
| | (10x stock) | Technologies | total volume) | |
| a | Primer No. 23 | | 4.5 mM | 450 nM |
| b | Primer No. 24 | | 4.5 mM | 450 nM |
| c | Probe No. 25 | | 2 mM | 200 nM |

TABLE 6
PCR reaction cycles

| No. | Temperatures | Duration | Cycles | Description |
|---|---|---|---|---|
| 1 | 95° C. | 360 s | 1x | Initial denaturation |
| 2 | 95° C. | 5 s | 45x | Denaturation |
| 3 | 60° C. | 30 s | | Annealing, Elongation |
| | | | | and Data capture |

LAMP reactions targeting TNF and ATI gene of MPXV can be performed in two methods: (1) “wet method” was based on NEB WARMSTART® Colorimetric LAMP 2× Master Mix (DNA & RNA); and (2) “dry method” was based on Haigene Bst 4.0 red/pH beads (A3828-01R). Initially, fluorescence LAMP dye (NEB) was added to both “wet” and “dry” systems with 1× concentration (2% total volume/0.5 μL and 0.4 μL per reaction for “wet” and “dry” systems, respectively). Hapten-tagged TNF and ATI LAMP primers (Table 1) in 10× concentrations were added into the reaction (10% total volume/2.5 μL and 2 μL per reaction for wet and dry system, respectively). Next, 5 μL of diluted positive control plasmid containing TNF and ATI gene (Table 3) was added into each positive control sample, then RNAse-free dH2O was added until the final reaction volume reached 25 μL. All components and the sample were mixed well before incubation. Detailed components and mixture for the “wet” and “dry” methods for both primer sets can be found in Tables 7 and 8. The LAMP reaction was preferably done in BioRad CFX Connect PCR instrument, with the following parameters: 63° C. reaction temperature, 45 mins total reaction, and SYBR channel for fluorescence reading (Table 8).

TABLE 7
Basic TNF LAMP amplification components

| | | | | |
|---|---|---|---|---|
| | | Supplier/ | | Final |
| | | Manu- | Mixed | concentration |
| No. | Reagents | facturer | volume | per reaction |

LAMP components: Wet reaction

| 1 | Mastermix | NEB | 12.5 μL | 1x | |
|---|---|---|---|---|---|
| | (WARMSTART ® | | (50% total | | |
| | Colorimetric | | volume) | | |
| | LAMP 2X Master | | | | |
| | Mix (DNA&RNA)) | | | | |
| 2 | LAMP Fluorescence | NEB | 0.5 μL | 1x | |
| | Dye (50X) | | (2% total | | |
| | | | volume) | | |
| 3 | Primer mix | Synbio | 2.5 μL | 1x | |
| | (10x stock) | Technologies | (10% total | | |
| | | | volume) | | |
| a | Primer No. 1 | | N/A | 1.6 | μM |
| b | Primer No. 2 | | | 0.12 | μM |
| c | Primer No. 3 | | | 1.6 | μM |
| d | Primer No. 4 | | | 0.12 | μM |
| e | Primer No. 5 | | | 0.4 | μM |
| f | Primer No. 6 | | | 0.4 | μM |

LAMP components: Dry reaction

| 1 | Haigene Bst 4.0 | Haigene | | 1 | bead |
|---|---|---|---|---|---|
| | red/pH beads | | | | |
| | (A3828-01R) | | | | |
| 2 | LAMP Fluorescence | NEB | 0.4 μL | 1x | |
| | Dye (50X) | | (2% total | | |
| | | | volume) | | |
| 3 | Primer mix | Synbio | 2 μL | 1x | |
| | (10x stock) | Technologies | (10% total | | |
| | | | volume) | | |
| a | Primer No. 1 | | N/A | 1.6 | μM |
| b | Primer No. 2 | | | 0.12 | μM |
| c | Primer No. 3 | | | 1.6 | μM |
| d | Primer No. 4 | | | 0.12 | μM |
| e | Primer No. 5 | | | 0.4 | μM |
| f | Primer No. 6 | | | 0.4 | μM |

TABLE 8
LAMP reaction settings (Using Biorad CFX Connect)

| | Temper- | Dura- | Cy- | |
|---|---|---|---|---|
| No. | atures | tion | cles | Description |
| 1 | 63° C. | 50 s | 45x | 1 cycle is close to 1 minute, |
| | | | | bringing total reaction time |
| | | | | to ~45 minutes |
| 2 | 63° C. | 2 s | | The fluorescence value is recorded |
| | | | | during this step with SYBR channel |

Consecutively, the same positive control samples were tested with qPCR concurrently. 5 μL (25% total reaction volume) Quantabio's ULTRAPLEX™ 1-step TOUGHMIX® 4× was mixed with 2 μL primers and probe mix (Table 5), 5 μL positive control sample with various dilution from 100 thousand to 10 million dilution, and diluted to 20 μL with RNAse-free dH₂O. Each sample dilution was prepared in three replicates with the cycle details set forth in Table 6. The average Ct value for each of the samples used are shown in Table 9. As the positive control sample contained both TNF and ATI sequences (Table 3), the qPCR would quantify for both TNF and ATI testing.

TABLE 9

| Dilution | Ct value (Average 3 tests) |
|---|---|
| 1:100K | 27.44756 |
| 1:1M | 31.74522 |
| 1:5M | 34.84511 |
| 1:10M | 35.591 |

Basic TNF primer sets defining the core LAMP-based amplification were first screened for optimal reaction conditions and verified using gel electrophoresis for characteristics ladder-like amplification typical of a successful LAMP reaction (FIG. 3). A total of 7 basic primer sets (without Loop Forward (LF) and Backward (LB)) were tested using 1:100 k diluted positive control plasmid. A reaction temperature of 63 and 65° C. were shown to have better amplification activity for most of the newly designed TNF LAMP primers (FIG. 3). Simultaneously, it can be seen that several primer sets (P1, P3 and P5) did not show any amplification for all temperatures tested suggesting sub-optimal performance of the primer design software. Likewise, P4 and P7 have positive results in the NTC samples, indicating false positive results came from these primer sets. Thus, only 2 primer sets, namely P2 and P10, were used for further study and design.

The basic primer sets P2 and P10 were then further tested with fluorimetric method in 63 and 65° C. reaction temperature using 1:100 k diluted positive control plasmid. Basic primer 2 was prepared according to Table 7 information for LAMP optimization. FIG. 4 shows that the P2 primer set was more efficient compared to P10 in both reaction temperatures for the said primer composition. P10 in 65° C. failed to amplify the sample, while P2 was only able to amplify 1 out of 2 samples. In 63° C., P2 successfully amplified both samples and achieved it in faster time (lower Cq of 27) compared to P10. Next, both primer sets were tested in 2 different sample concentrations (1:100 k and 1:1M dilution). Similarly, the P2 primer set performed better in both concentrations, with statistical difference (P value of 0.002). Based on this result, the P2 primer set showed better potential for the MPXV detection and was selected for further optimization and design.

Primer set P2 then improved by adding Loop primers forward (LF; SEQ ID No: 5) and backwards (LB; SEQ ID No: 6). Tests were made using 1:100 k diluted plasmid and the speed of the reaction was compared between the basic P2 set (Table 7) and addition of either loop primers or both. Addition one loop primer reduces the reaction time (Cq value) considerably, but addition of both loop primers have a more pronounced effect and cuts the reaction time by half (FIG. 5). Statistical test (t-test) also showed the addition of both loop primers significantly affect the reaction speed.

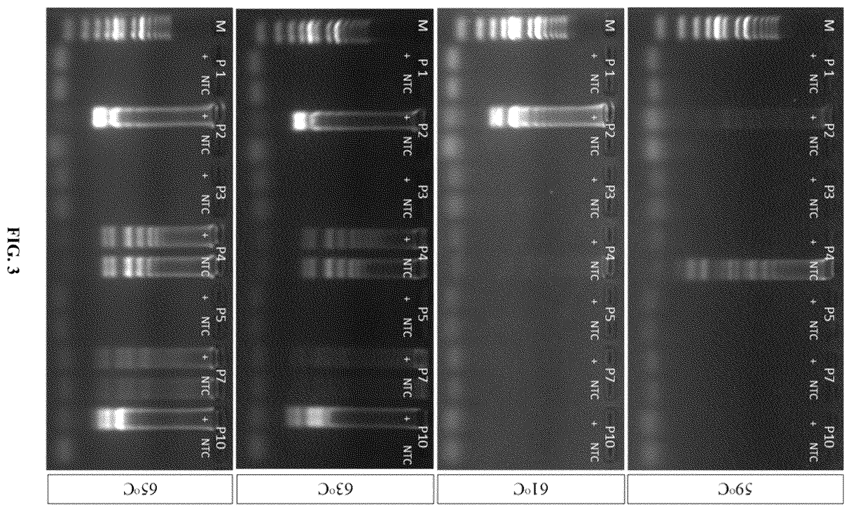

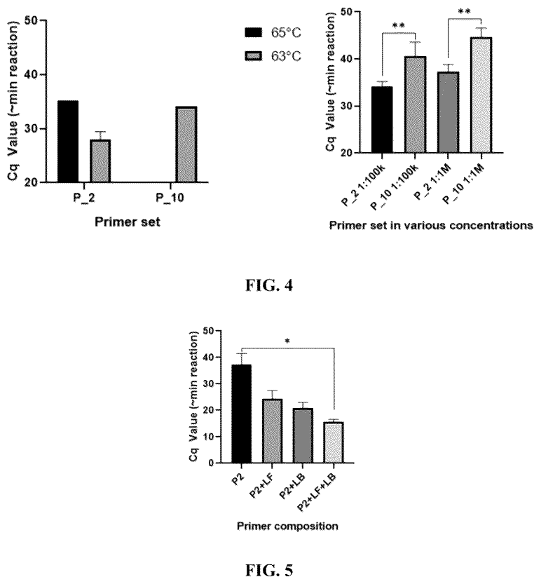

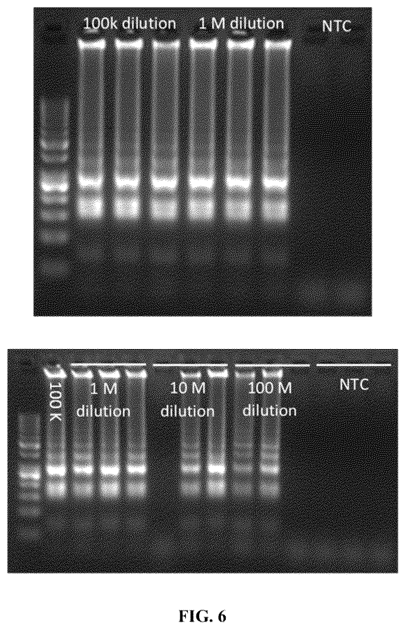

Next, positive control samples diluted from 1:100 k times to 1:100M times were tested on a full P2 TNF system and visualized on the gel electrophoresis. As seen in FIG. 6, the P2 primer set is shown to be able to detect up to 100 M dilution sample, or one magnitude lower than 10 M dilution, or equivalent to roughly 35.6 Ct value based on a standard qPCR setting (Table 9). Therefore, P2 primer set have good sensitivity for detecting MPXV TNF sequence.

For the next part, a primer tagging matrix was designed to aid screening of LFA-compatible TNF primer and screen the best set of primer combinations for the Real-time LFA-based LAMP analysis. Primer tagging matrix was based on tagging 4 out of 6 primers in the TNF LAMP set, excluding the forward and backward outer primers (F3 and B3), with Biotin and FAM (Table 1, SEQ ID Nos: 7-12). Through a combination of 4 primers and 2 tags, with an assumption that the reverse pairing (e.g. FIP-Bio with LF-FAM and FIP-FAM with LF-Bio) are the same, a total of 6 different combinations of primer sets with unique tagging-identity (henceforth identified as Method 6 to Method 11) were systematically screened. The reaction was performed with NEB WARMSTART® LAMP Kit (DNA & RNA) master mix. Sample used is positive control plasmid with TNF sequence (Table 3, SEQ ID No: 22), diluted 1:100 k for the test. Tests were performed on Bio-Rad CFX Connect with the reaction components for each combination described in Table 10.

TABLE 10
TNF LAMP components for Primer Tagging test

| | | Supplier/ | Mixed | Final | |
|---|---|---|---|---|---|
| | | Manu- | volume | concentration | |
| No. | Reagents | facturer | (if any) | per reaction | |
| 1 | Mastermix | NEB | 12.5 μL | 1x | |
| | (WARMSTART ® | | (50% total | | |
| | LAMP Kit | | volume) | | |
| | (DNA&RNA)) | | | | |
| 2 | LAMP Fluorescence | NEB | 0.5 μL | 1x | |
| | Dye (50X) | | (2% total | | |
| | | | volume) | | |
| 3 | Sample | IDT | 5 μL | 1:100k dilution | |
| | | | (25% total | | |
| | | | volume) | | |
| 4 | dH₂O | | To 25 μL | — | |
| 5 | Primer mix | Synbio | 2.5 μL | 1x | |
| | (10x stock) | Technologies | (10% total | | |
| | | | volume) | | |
| | Method 6 (M6) | | | | |
| a | Primer No. 1 | | N/A | 1.6 | μM |
| b | Primer No. 2 | | | 0.12 | μM |
| c | Primer No. 8 | | | 1.6 | μM |
| d | Primer No. 4 | | | 0.12 | μM |
| e | Primer No. 5 | | | 0.4 | μM |
| f | Primer No. 12 | | | 0.4 | μM |
| | Method 7 (M7) | | | | |
| a | Primer No. 7 | | N/A | 1.6 | μM |
| b | Primer No. 2 | | | 0.12 | μM |
| c | Primer No. 3 | | | 1.6 | μM |
| d | Primer No. 4 | | | 0.12 | μM |
| e | Primer No. 11 | | | 0.4 | μM |
| f | Primer No. 6 | | | 0.4 | μM |
| | Method 8 (M8) | | | | |
| a | Primer No. 7 | | N/A | 1.6 | μM |
| b | Primer No. 2 | | | 0.12 | μM |
| c | Primer No. 3 | | | 1.6 | μM |
| d | Primer No. 4 | | | 0.12 | μM |
| e | Primer No. 5 | | | 0.4 | μM |
| f | Primer No. 12 | | | 0.4 | μM |
| | Method 9 (M9) | | | | |
| a | Primer No. 1 | | N/A | 1.6 | μM |
| b | Primer No. 2 | | | 0.12 | μM |
| c | Primer No. 8 | | | 1.6 | μM |
| d | Primer No. 4 | | | 0.12 | μM |
| e | Primer No. 11 | | | 0.4 | μM |
| f | Primer No. 6 | | | 0.4 | μM |
| | Method 10 (M10) | | | | |
| a | Primer No. 1 | | N/A | 1.6 | μM |
| b | Primer No. 2 | | | 0.12 | μM |
| c | Primer No. 3 | | | 1.6 | μM |
| d | Primer No. 4 | | | 0.12 | μM |
| e | Primer No. 9 | | | 0.4 | μM |
| f | Primer No. 11 | | | 0.4 | μM |
| | Method 11 (M11) | | | | |
| a | Primer No. 10 | | N/A | 1.6 | μM |
| b | Primer No. 2 | | | 0.12 | μM |
| c | Primer No. 8 | | | 1.6 | μM |
| d | Primer No. 4 | | | 0.12 | μM |
| e | Primer No. 5 | | | 0.4 | μM |
| f | Primer No. 6 | | | 0.4 | μM |

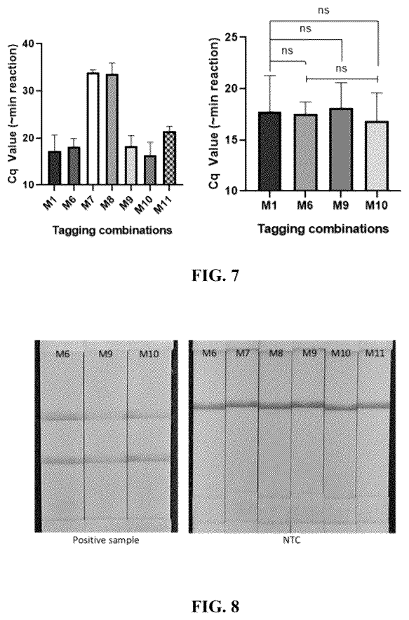

The Cq values of each unique tagging option can be seen in Table 11 and displayed in FIG. 7. Cq values of each primer tagging combinations describe the efficiency of the amplification, with the lower Cq correlates with faster and more efficient amplification. Test using 1:100 k sample dilution (equivalent to 27.4 Ct value in qPCR/Table 9) shows significant performance variations among the combinations. M6, M9 and M10 have comparable Cq values of 18.22, 16.04 and 14.59, respectively. The other 3 methods have much higher Cq values, signifying these primer modifications adversely impacted the amplifications.

TABLE 11

| Primer-Tag combinations | Average Cq | StDev |
|---|---|---|
| Method 1 | 15.78 | 0.35 |
| Method 6 | 18.22 | 1.54 |
| Method 7 | 33.92 | 0.60 |
| Method 8 | 33.67 | 2.26 |
| Method 9 | 16.04 | 1.46 |
| Method 10 | 14.59 | 0.60 |
| Method 11 | 21.49 | 1.02 |

Comparing the TNF primer set with and without hapten tags showed that addition of hapten tags does not significantly affect the sensitivity of the system (FIG. 7). Despite the difference in Cq values across the methods, there are no statistical differences between M6 (t test P value 0.624), M9 (P value 0.5679) and M10 (P value 0.5753) with M1. LFA tests on the samples were performed on all samples NTCs and 3 best methods (M6, M9 and M10), which do not give statistical difference between each other (1-way ANOVA test P value 0.3085) All NTCs result in FIG. 8 showed negative LFA results, which indicates all primer tags that do not cause false positive results can potentially be used in LFA-LAMP systems. Meanwhile, the positive samples test showed all 3 top primer tags combinations showed positive results. Therefore, these 3 methods are the best combinations for LAMP-LFA use. For further verification and proof-of real-time LFA concept, however, M6 primer was randomly selected in the subsequent experiment.

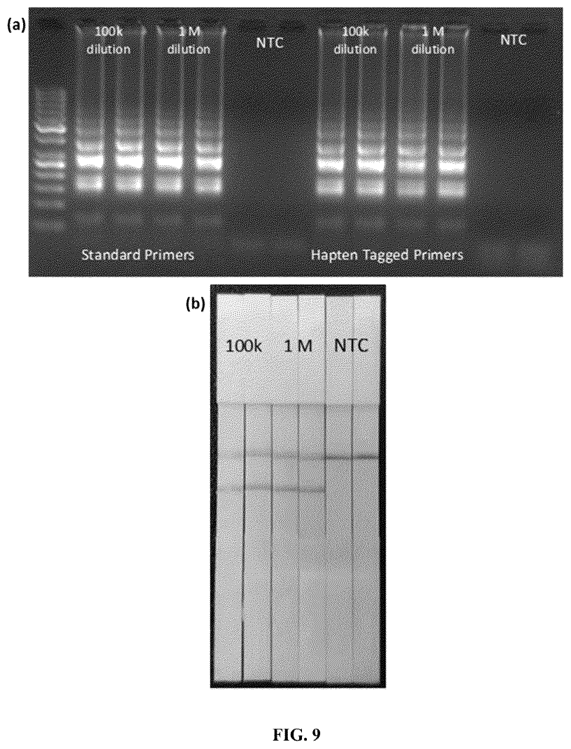

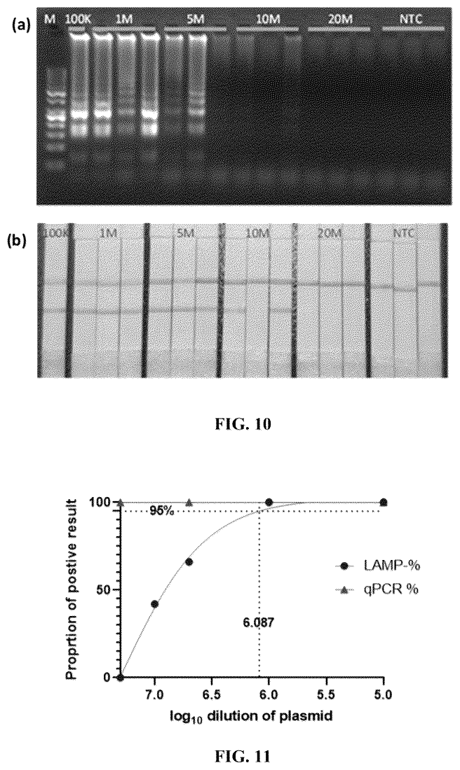

The LoD of the TNF LAMP-LFA system was assessed by running the system on various sample concentrations. Initial test on 1:100 k and 1:1M dilution (FIG. 9) shows gel electrophoresis and LFA results of the reactions. The tagged primers visualization in gel gives positive results in accordance with the gel electrophoresis result, showing positive in all samples. Next, the system is checked with additional dilutions of positive control plasmid (1:100 k to 1:20M dilutions) were used to measure the LoD of the system. Table 12 showed the average Cq value for each sample and positive rate over several technical and biological replicates. Average Cq value increases as the sample was more diluted, and the reaction started to have false negative results in 5 and 10 million dilution, or roughly 35-36 Ct value equivalent in qPCR (Table 9). LFA test result and its positive rate (FIG. 10) showed the system can detect up to 1:10M diluted samples, with 2 out of 3 samples being detected. Probability plot analysis on the result showed the approximate LoD of the system is in 6.087 log 10 dilution sample, or roughly 1.221M times dilution (FIG. 11).

TABLE 12

| Sample (dilutions) | Average Cq | Positive rate | LFA positive rate |
|---|---|---|---|
| 1:100k | 27.21 | 100% | 1/1 |
| 1:1M | 30.84 | 100% | 3/3 |
| 1:5M | 38.28 | 66.7% | 3/3 |
| 1:10M | 37.71 | 42.8% | 2/3 |
| 1:20M | N/A | 0% | 0/3 |

To investigate the specificity of LAMP primer with the optimized LAMP-LFA tag for TNF gene (M6), closely and distantly related non-MPXV orthopox virus, bacterial species that potentially contaminate samples and human DNA from cell lines were tested. Plasmids containing non-MPXV orthopox virus sequences were outsourced to Synbio Technologies. The full list of samples used in the specificity test is listed in Table 13.

The LFA and gel electrophoresis results showed that the primer set was able to specifically amplify the target DNA sequence of TNF, but not the human, bacterial or plasmids containing viral DNA sequences under the test. Therefore, the primer set has good selectivity towards MPXV. One exception is a false positive result for Variola virus, a causative agent of smallpox disease. However, as the virus is declared eradicated through WHO Smallpox Global Eradication Programme, (Meyer et al., “Smallpox in the Post-Eradication Era”, Viruses 2020, 12(2), 138) the false positive may pose little clinical impact on detection of MPXV.

TABLE 13
Bacteria species and non-MPXV poxvirus used for
testing cross reactivity of the LAMP assay.

| Samples | | Ref. No (if any) | Result* |
|---|---|---|---|
| 1 | Klebsiella pneumonia | ATCC78578 | Negative |
| 2 | Klebsiella pneumonia | ATCC13883 | Negative |
| 3 | Acinetobacter | ATCC19606 | Negative |
| 4 | Pseudomonas protegens | ATCC BAA-477TM | Negative |
| 5 | E. coli | ATCC25922 | Negative |
| 6 | SARS-COV2_N-gene | IDT 10006625 | Negative |
| 7 | HeLa Cell line DNA | N/A | Negative |
| 8 | Variola virus, complete | >NC_001611.1/182624- | Positive |
| | genome | 182821 | |
| 9 | Vaccinia virus strain IOC | >KT184690.1/188497- | Negative |
| | clone B141, complete genome | 188670 | |
| 10 | Rabbitpox virus, complete | >AY484669.1/193979- | Negative |
| | genome | 194161 | |
| 11 | Camelpox virus strain | >MK910851.1/199434- | Negative |
| | Negev2016, complete genome | 199631 | |
| 12 | Taterapox virus, complete | >NC_008291.1/195330- | Negative |
| | genome | 195527 | |
| 13 | Cowpox virus strain Brighton | >AF482758.2/219907- | Negative |
| | Red, complete genome | 220110 | |

*Results were run in triplicate in two independent experiments.

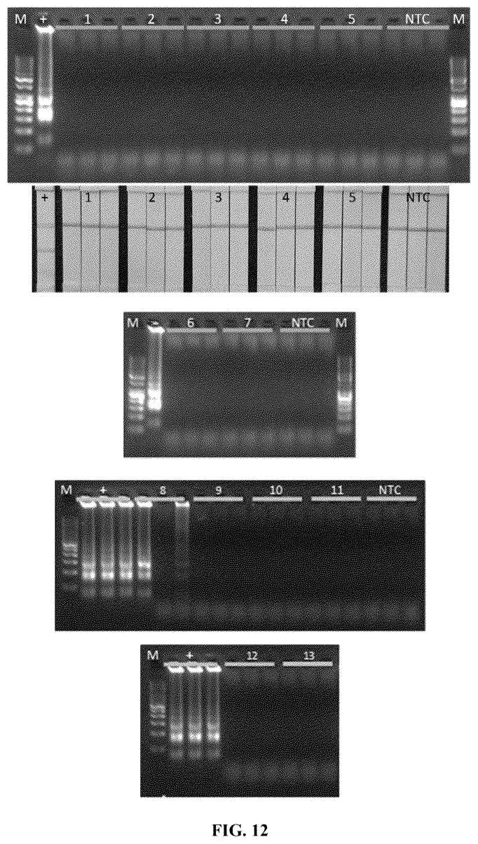

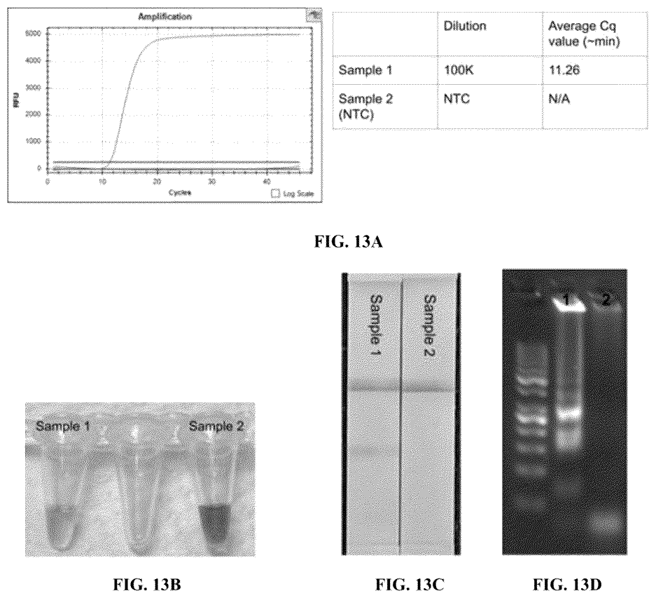

Real-time LAMP then tested using the optimized TNF primer set. FIG. 13 illustrates an example of using a sample with a hundred thousand (1:100K) times dilution of MPXV plasmid as a positive control (sample 1) and a no-template control (NTC) (sample 2) to incubate with the reaction mixture including LAMP primers specific for TNF-α receptor gene (TNF LAMP primers), phenol red as colorimetric dye, and fluorescence dye. An antibody specific to the biotin- and FAM-tags in the amplicons generated by the TNF LAMP primers modified with the corresponding tags at their 5′-end is also included into the reaction mixture. FIG. 13A is a plot of fluorescence signal detected throughout the LAMP reaction, and the table as shown in the inset summarizes the dilution factor of positive control template and their corresponding quantification cycle (Cq) value. The instrument was set to have 1 minute per cycle. As seen in FIG. 13A, an average Cq value of positive control sample is about 11.26 minutes, indicated by a spiked fluorescence signal in the curve. FIG. 13B shows an image of colorimetric assay on samples 1 and 2 (positive and negative controls, respectively) obtained at the end of the LAMP reaction indicating a change in color in the positive control sample but no color change in the negative control. The color change from red into yellow represents the presence of amplicons of the target sequence, and the degree of color change may be in accordance with an increase in the number of thermocycles. The full LAMP reaction was set for 45 minutes, so there will be about 45 thermocycles completed. The Cq value of a LAMP reaction correlates to a duration of reaction, i.e., the duration of which a significant number of amplicon copies is generated. In this example, the average Cq value is about 11.26. Assuming each thermocycle takes about 1 minute, to reach a positive fluorimetric result, the LAMP reaction is only required to take less than 12 minutes instead of 45 minutes to assure that a test sample (with 1:100K dilution of a target sequence) is positive, thereby shortening the LAMP reaction time. LFA and gel electrophoresis results of the 2 samples (FIGS. 13C and 13D) confirmed the amplification result.

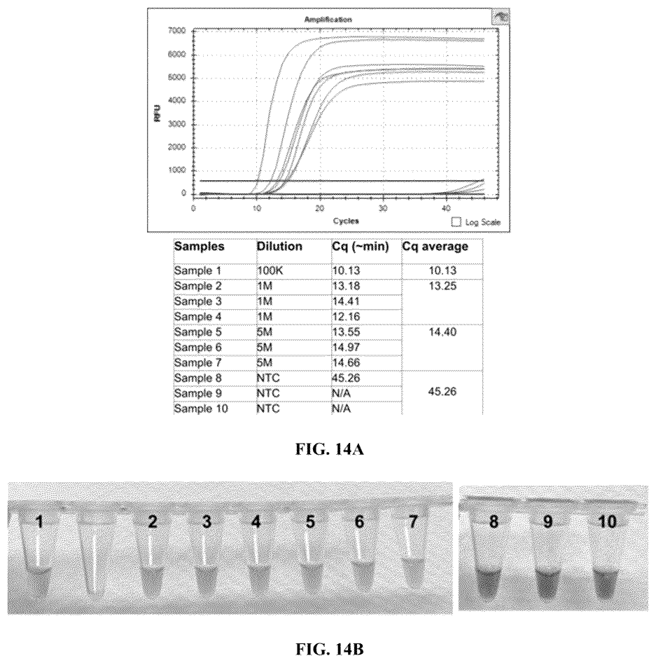

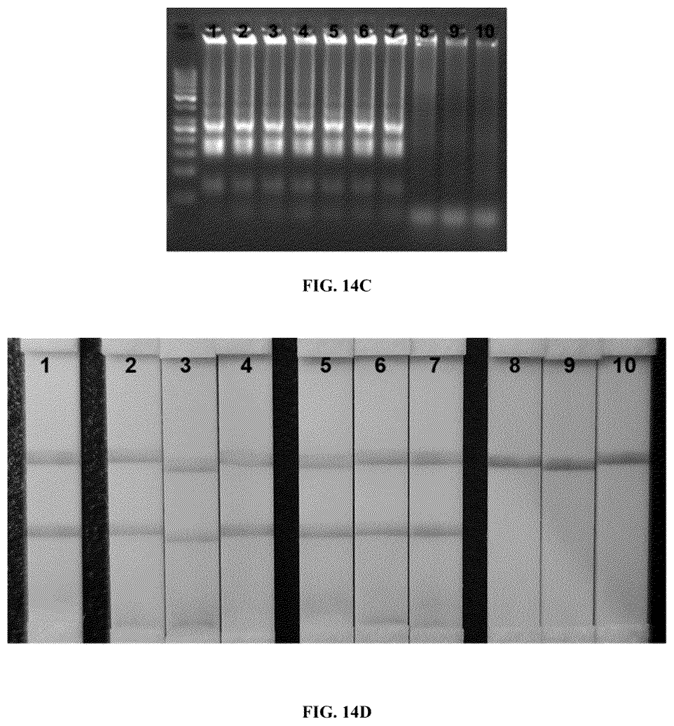

FIGS. 14A-14D illustrate a consistency test result on the one-pot reaction mixture of the present pathogen detection system for positive control samples in various concentrations in terms of the same set of assays used in the example of FIG. 13. Samples used in this example were prepared in several concentrations based on dilution of the plasmid sample, ranging from 100 thousand, 1 million, 5 million and 10 million dilutions, with Ct values roughly 27.4; 31.7; 34.8; and 35.6 (Table 9). In FIG. 14A, the fluorescence emission curve shows that most of the reactions are finished in less than 15 minutes (signified by the Cq value not exceeding 15). However, at the end of the 45th minute, one of the NTC triplicates gave a false positive reading, and according to the curve, the remaining NTC samples might also provide false positive results if the reaction is prolonged. The false positive might come from non-specific amplification caused by amplification error after prolonged reaction.

FIG. 14B shows the color change of the colorimetric dye (i.e., phenol red) in various samples obtained by the end of the 45th minute, in which the positive control samples in different concentrations (sample nos. 1-7) yielded yellow-color result, whereas the NTC samples (sample nos. 8-10) remained reddish. Furthermore, the gel electrophoresis result as shown in FIG. 14C shows the bands in lanes 1-7 (corresponding to sample nos. 1-7), which exhibited formation of certain reaction products (i.e., amplicons), whereas lanes 8-10 did not give obvious, solid bands, but only some faint streak bands. These gel electrophoresis results demonstrate the specificity problem in many common LAMP result interpretations, since the presence of faint streak bands is an indication of a susceptible or non-specific amplification reaction in the system. Relying on these methods alone could easily lead to false positive results.

Therefore, an additional LFA was carried out to validate the findings in the colorimetric, fluorimetric and gel electrophoresis analyses. FIG. 14D shows two bands in samples nos. 1-7 (positive control samples), whereas only one band was present in samples nos. 8-10 (the NTC samples).

As seen in FIGS. 13A-13D and 14, through a combination of several result interpretation methods, it is possible to provide a hybrid real-time LAMP test with high specificity. By using fluorescence or color changes as the indicator of the potential positive result, followed by a LFA to check whether the amplification is directed to the desired target or not, the LAMP reaction time can be significantly reduced while maintaining the high specificity by virtue of the LFA result.

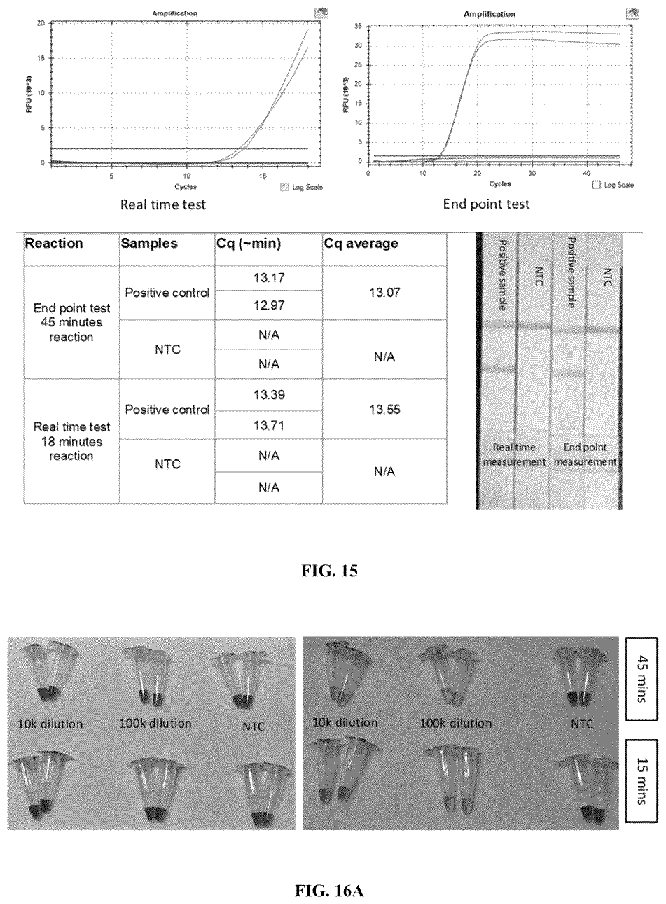

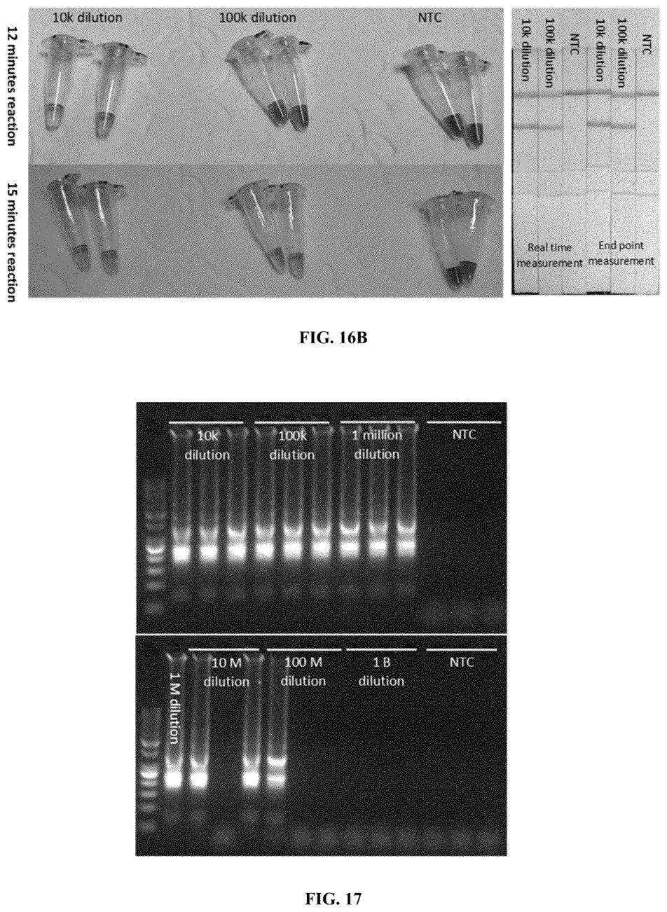

FIGS. 15 and 16A-16B respectively show the difference in fluorimetric aspect of the positive and negative control samples between those obtained at the end-point of the reaction and those obtained real-time (i.e., obtained at which a threshold number of thermocycles indicated by the corresponding Cq value of each sample), and the colorimetric aspect of those samples obtained real-time, both being validated by the corresponding LFA result. In FIG. 15, the upper panel shows the fluorescence signal curves for positive control samples obtained real-time (upper left inset) and at the end-point (upper right inset) of the LAMP reaction. The positive control samples contained 10K diluted MPXV plasmids and the end point was obtained at 45 minutes. LAMP reaction conditions can be referred to Table 8.

As shown in the table of FIG. 15, the positive results indicated by a spiked emission of fluorescence signal occurred at around 13 minutes (in terms of the actual and average Cq value), notwithstanding the samples obtained real-time (obtained at about 18 minutes in this example) or those obtained at the end point (i.e., ˜45 minutes). The corresponding LFA result as shown in FIG. 15 verifies the positive results obtained in the fluorimetric assay (indicated by two bands in both real-time and end-point samples). These results suggest that the shortened LAMP reaction in terms of the Cq value obtained in the fluorimetric assay does not affect the specificity. In contrast, redundant thermocycles during LAMP reaction after reaching the Cq value increases the likelihood of having false positive results from no-template (no target sequence) controls.

FIGS. 16A and 16B further show the difference in color change in 1:10K and 1:100K diluted positive control samples obtained at different time points around the Cq value according to the results as shown in FIG. 15 and also at the end point of the LAMP reaction. In FIG. 16A, the initial red color (in left panel) turned into yellowish color in both 1:10K and 1:100K diluted positive samples at both 15th minute and the end point (i.e., 45th minute), whereas the no-template controls (NTC) remained reddish at both 15th minute and the end point. Images in FIG. 16B further show the color change difference between the positive samples obtained at 12th and 15th minutes of the LAMP reaction. At 12th minute (images in upper row), the positive samples in 1:10K dilution had a significant color change into yellowish, whereas the positive samples in 1:100K dilution did not have an obvious observable yellowish color change. At 15th minute (images in lower row), on the other hand, both 1:10K and 1:100K diluted positive samples had significant color change into yellowish, while the NTC samples obtained at both 12th and 15th minute remained reddish. Corresponding LFA results as shown in the right panel of FIG. 16B demonstrate two bands in both 1:10K and 1:100K diluted positive samples obtained real-time (at 12th minute for 1:10K dilution versus at 15th minute for 1:100K dilution) and at the end point, verifying the results obtained in the colorimetric assay. These results further suggest that the incorporation of the LFA interpretation significantly reduces the LAMP reaction time, thereby improving the detection efficiency and also the specificity of the result.

Similarly, the detection of ATI gene of MPXV was performed with ATI LAMP primer set using both “wet” and “dry” methods with NEB's WARMSTART® Colorimetric LAMP 2× Master Mix (DNA&RNA) and Haigene's Bst 4.0 red/pH beads (A3828-01R), respectively, based on ATI LAMP primers set forth in Table 1. ATI primer sequence was cited from (Iizuka et al., “Loop-mediated isothermal amplification-based diagnostic assay for monkeypox virus infections”, J Med Virol. 2009 June; 81(6): 1102-8) and tested with ATI samples listed in Table 3. The initial screening reaction was performed in accordance with the mixture concentration and conditions set forth in Table 14.

The same positive control samples were concurrently tested with qPCR to measure the Ct value of samples used in the test. 5 μL (25% total reaction volume) Quantabio's ULTRAPLEX™ 1-step TOUGHMIX® 4× was mixed with 2 μL primers and probe mix (Table 5). a 5 μL positive control sample with various dilutions from 1:100K to 1:10M times, each with three replicates, was diluted to 20 μL with RNAse-free dH₂O. Corresponding average Ct value for the samples at each dilution could be referred to Table 9.

TABLE 14
Basic ATI LAMP amplification components

| | | | | |
|---|---|---|---|---|
| | | Supplier/ | Mixed | Final |
| | | Manufac- | volume | concentration |
| No. | Reagents | turer | (if any) | per reaction |

LAMP components: Wet reaction

| 1 | Mastermix | NEB | 12.5 μL | 1x | |
|---|---|---|---|---|---|
| | (WARMSTART ® | | (50% total | | |
| | Colorimetric LAMP | | volume) | | |
| | 2X Master Mix | | | | |
| | (DNA&RNA)) | | | | |
| 2 | LAMP Fluorescence | NEB | 0.5 μL | 1x | |
| | Dye (50X) | | (2% total | | |
| | | | volume) | | |
| 3 | Primer mix | Synbio | 2.5 μL | 1x | |
| | (10x stock) | Technologies | (10% total | | |
| | | | volume) | | |
| a | Primer No. 13 | | N/A | 1.6 | μM |
| b | Primer No. 14 | | | 0.12 | μM |
| c | Primer No. 15 | | | 1.6 | μM |
| d | Primer No. 16 | | | 0.12 | μM |
| e | Primer No. 17 | | | 0.4 | μM |
| f | Primer No. 18 | | | 0.4 | μM |

TABLE 15
ATI LAMP components for Primer Tagging and LFA tests

| | | | | |
|---|---|---|---|---|
| | | Supplier/ | Mixed | Final |
| | | Manufac- | volume | concentration |
| No. | Reagents | turer | (if any) | per reaction |

LAMP components: Wet reaction

| 1 | Mastermix | NEB | 12.5 μL | 1x | |
|---|---|---|---|---|---|
| | (WARMSTART ® | | (50% total | | |
| | LAMP Kit | | volume) | | |
| | (DNA&RNA)) | | | | |
| 2 | LAMP Fluorescence | NEB | 0.5 μL | 1x | |
| | Dye (50X) | | (2% total | | |
| | | | volume) | | |
| 3 | Sample | IDT | 5 μL | 1:1M to 1:5M | |
| | | | (25% total | dilution | |
| | | | volume) | | |
| 4 | dH₂O | | To 25 μL | — | |
| 5 | Primer mix (10x stock) | Synbio | 2.5 μL | 1x | |
| | | Technologies | (10% total | | |
| | | | volume) | | |
| a | Primer No. 13 | | N/A | 1.6 | μM |
| b | Primer No. 14 | | | 0.12 | μM |
| c | Primer No. 19 | | | 1.6 | μM |
| d | Primer No. 16 | | | 0.12 | μM |
| e | Primer No. 17 | | | 0.4 | μM |
| f | Primer No. 20 | | | 0.4 | μM |

LAMP components: Dry reaction

| 1 | Haigene Bst 4.0 red/ | Haigene | N/A | 1 | bead |
|---|---|---|---|---|---|
| | pH beads (A3828- | | | | |
| | 01R) | | | | |
| 2 | LAMP Fluorescence | NEB | 0.4 μL | 1x | |
| | Dye (50X) | | (2% total | | |
| | | | volume) | | |
| a | Primer No. 13 | Synbio | N/A | 1.6 | μM |
| | | Technologies | | | |
| b | Primer No. 14 | Synbio | | 0.12 | μM |
| | | Technologies | | | |
| c | Primer No. 19 | Synbio | | 1.6 | μM |
| | | Technologies | | | |
| d | Primer No. 16 | Synbio | | 0.12 | μM |
| | | Technologies | | | |
| e | Primer No. 17 | Synbio | | 0.4 | μM |
| | | Technologies | | | |
| f | Primer No. 20 | Synbio | | 0.4 | μM |
| | | Technologies | | | |

The initial testing result of the untagged ATI primer set on positive control samples is shown in FIG. 17 based on Table 14, indicating the original primer set can detect up to 1 million times of dilution consistently in all samples tested. Although the primer set can further detect samples diluted up to 100 million (1:100M), the gel electrophoresis analysis reveals that it is inconsistent among the triplicates.

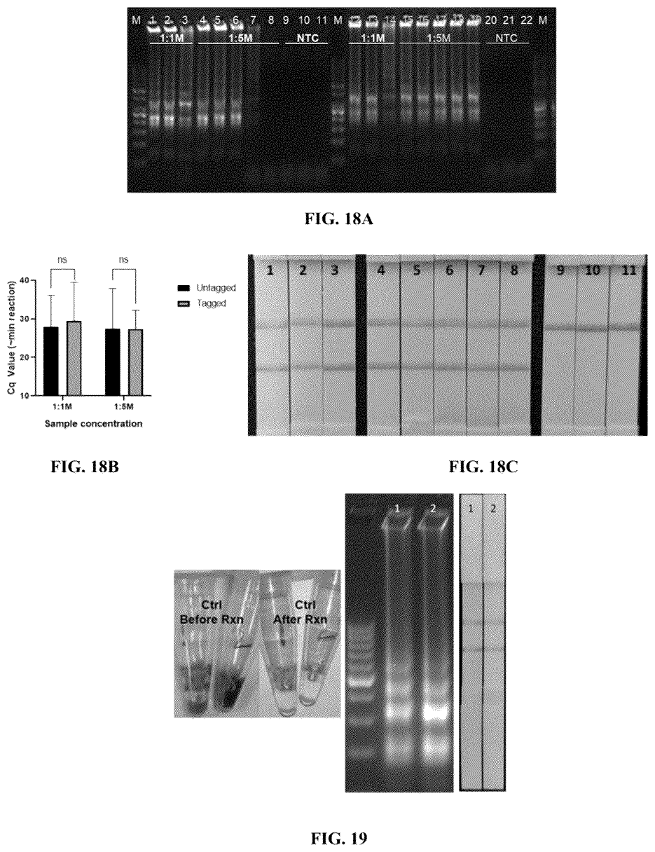

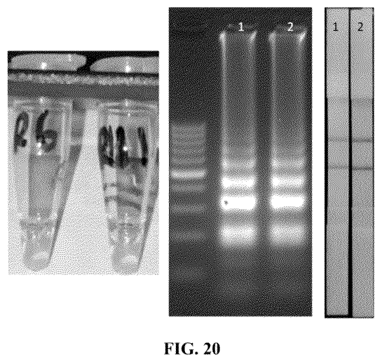

Next, ATI primer set was modified for LFA adaptation by tagging 2 hapten tags into 2 of the primers, as written in Table 15. Tests on tagged ATI primer set on positive control up to 5 million times dilution (1:5M or approximately 34.8 Ct value; Table 9) showed persistent detection on all samples (FIG. 18). Table 16 and FIG. 18A showed that modified ATI primer set has no effect on the performance of the primer set. Statistical test (t-test) showed no difference in the Cq value for modified primers compared to untagged primers (FIG. 18B). LFA test on the samples (FIG. 18C) shows all samples are in accordance with the fluorescent and gel electrophoresis result. The colorimetric and LFA test results on 1:100K (FIG. 19) and 1:1M (FIG. 20) dilution samples demonstrate the synergy of the colorimetric and LFA method, giving potential for real-time LAMP test with high specificity.

TABLE 16

| Primer | Lane | Dilution | Cq (~min) | Cq average |
|---|---|---|---|---|
| Untagged | 1 | 1M | 21.81 | 27.99 |
| ATI | 2 | 1M | 24.94 | |
| Primers | 3 | 1M | 37.21 | |
| | 4 | 5M | 20.68 | 27.4 |
| | 5 | 5M | 21.79 | |
| | 6 | 5M | 24.05 | |
| | 7 | 5M | 43.06 | |
| | 8 | 5M | N/A | |
| | 9 | NTC | N/A | N/A |
| | 10 | NTC | N/A | |
| | 11 | NTC | N/A | |
| Tagged | 12 | 1M | 24.95 | 29.51 |
| ATI | 13 | 1M | 22.57 | |
| Primers | 14 | 1M | 41.01 | |
| | 15 | 5M | 30.32 | 27.366 |
| | 16 | 5M | 33.38 | |
| | 17 | 5M | 24.84 | |
| | 18 | 5M | 20.64 | |
| | 19 | 5M | 27.65 | |
| | 20 | NTC | N/A | N/A |
| | 21 | NTC | N/A | |
| | 22 | NTC | N/A | |

### EXAMPLE 2 (COVID-19 E GENE)

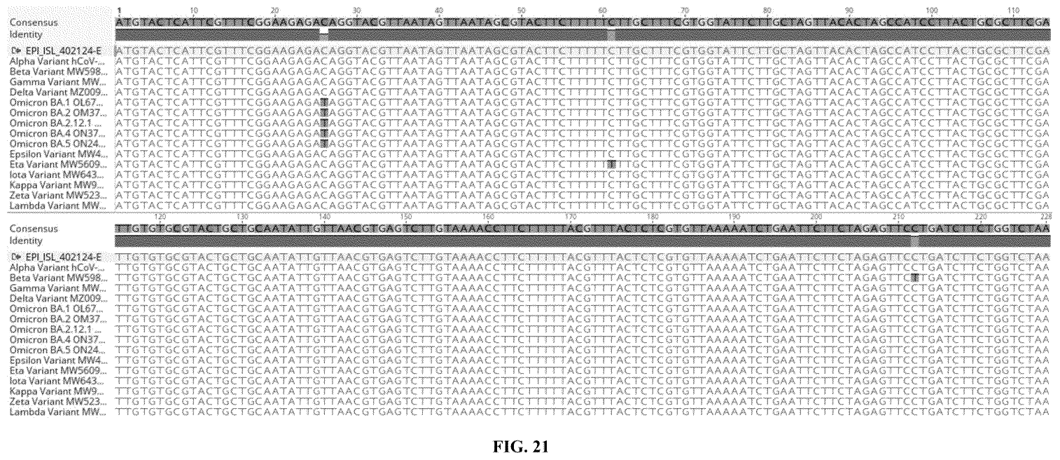

A system was developed to detect Covid-19 by targeting the E gene with the Real-time LFA system. E gene sequences of various SARS-COV-2 variants were aligned to check the mutations in the sequences in different variants of the virus. FIG. 21 displayed the alignment of the sequences. The top sequence is the reference sequence Wuhan-Hu-1 from the original outbreak (SEQ ID No: 49), aligned with the E gene sequence from each variant (Alpha, Beta, Gamma, and so on) (SEQ ID Nos: 97-111). Sub Variants of Omicron (BA.1, BA.2, BA.2.12.1, BA.4 and BA.5) (SEQ ID Nos: 101-105) were also included. The alignment showed that there are 3 nucleotide mutations in the E gene region throughout the SARS-COV-2 mutations from Alpha to Omicron variants. Mutation 26C>T, 61C>T and 212C>T are found scattered among the variants, with 26C>T is common in the most recent Omicron variant. However, most of the E region remains conserved and suitable as a diagnostic target.

In total, 3 E-gene LAMP primer sets were screened, with E1 primer set is based on reports by Broughton et al. (“Rapid Detection of 2019 Novel Coronavirus SARS-COV-2 Using a CRISPR-based DETECTR Lateral Flow Assay”, medRxiv., 2020 Mar. 27; 2020.03.06.20032334) and verified by Yang et al. (“Rapid and convenient detection of SARS-COV-2 using a colorimetric triple-target reverse transcription loop-mediated isothermal amplification method”, PeerJ. 10:e14121) and Dong et al (“Comparative evaluation of 19 reverse transcription loop-mediated isothermal amplification assays for detection of SARS-CoV-2”, Scientific Reports, 11, 2936 (2021)). Meanwhile, the other 2 primer sets (E2 and E3) were designed using PrimerExplorer based on the Wuhan-Hu-1 reference sequence. All primers were synthesized by IDT Technologies (Singapore) and Synbio Technologies (Suzhou, China). Set E1 and E2 consist of 6 primers, while E3 consists of 5 primers due to the small region of the E gene region. The full list of the primer sequences is listed in Table 17.

The positive control samples used in certain examples of the present disclosure for the detection of the presence of E gene sequence of SARS-COV-2 virus. The sequence for positive control was taken from consensus sequence of the E gene across multiple variants (FIG. 21 and Table 18) and produced in the same plasmid with the positive control of TNF and ATI of MPXV (Table 3). Alternatively, an E gene plasmid sample from IDT with 200000 copies/μL sample amount was used.

TABLE 17

| | | | | |
|---|---|---|---|---|
| SEQ | | | | |
| ID | | Primer | | |
| No | Target | name | Sequence | Modifications |

E1 primer

| | | | | |
|---|---|---|---|---|
| 26 | Covid- | F3_E1 | 5′-CCGACGACGACTACTAGC-3′ | N/A |
| | 19 E | | | |
| | gene | | | |
| 27 | Covid- | B3_E1 | 5′-AGAGTAAACGTAAAAAGAAGGTT-3′ | N/A |
| | 19 E | | | |
| | gene | | | |
| 28 | Covid- | FIP_E1 | 5′- | N/A |
| | 19 E | | ACCTGTCTCTTCCGAAACGAATTTGTAAGCACA | |
| | gene | | AGCTGATG-3′ | |
| 29 | Covid- | BIP_E1 | 5′- | N/A |
| | 19 E | | CTAGCCATCCTTACTGCGCTACTCACGTTAACA | |
| | gene | | ATATTGCA-3′ | |
| 30 | Covid- | LF_E1 | 5′-TCGATTGTGTGCGTACTGC-3′ | N/A |
| | 19 E | | | |
| | gene | | | |
| 31 | Covid- | LB_E1 | 5′-TGAGTACATAAGTTCGTAC-3′ | N/A |
| | 19 E | | | |
| | gene | | | |

E2 Primer set

| | | | | |
|---|---|---|---|---|
| 32 | Covid- | F3_E2 | 5′-TTTCGGAAGAGACAGGTAC-3′ | N/A |
| | 19 E | | | |
| | gene | | | |
| 33 | Covid- | B3_E2 | 5′-AGGAACTCTAGAAGAATTCAGA-3′ | N/A |
| | 19 E | | | |
| | gene | | | |
| 34 | Covid- | FIP_E2 | 5′- | N/A |
| | 19 E | | CGCAGTAAGGATGGCTAGTGTAGCGTACTTCT | |
| | gene | | TTTTCTTGCTT-3′ | |
| 35 | Covid- | BIP_E2 | 5′- | N/A |
| | 19 E | | TCGATTGTGTGCGTACTGCTGTTTTTAACACG | |
| | gene | | AGAGTAAACGT-3′ | |
| 36 | Covid- | LF_E2 | 5′-CTAGCAAGAATACCACG-3′ | N/A |
| | 19 E | | | |
| | gene | | | |
| 37 | Covid- | LB_E2 | 5′-GTTAACGTGAGTCTTG-3′ | N/A |
| | 19 E | | | |
| | gene | | | |
| 38 | Covid- | FIP_BIO_ | 5′- | 5′-Biotin |
| | 19 E | E2 | CGCAGTAAGGATGGCTAGTGTAGCGTACTTCT | labeled |
| | gene | | TTTTCTTGCTT-3′ | |
| 39 | Covid- | BIP_BIO_ | 5′- | 5′-Biotin |
| | 19 E | E2 | TCGATTGTGTGCGTACTGCTGTTTTTAACACGA | labeled |
| | gene | | GAGTAAACGT-3′ | |
| 40 | Covid- | LB_BIO_ | 5′-GTTAACGTGAGTCTTG-3′ | 5′-Biotin |
| | 19 E | E2 | | labeled |
| | gene | | | |
| 41 | Covid- | FIP_ | 5′- | 5′-FAM |
| | 19 E | FAM_E2 | CGCAGTAAGGATGGCTAGTGTAGCGTACTTCT | labeled |
| | gene | | TTTTCTTGCTT-3′ | |
| 42 | Covid- | LF_ | 5′-CTAGCAAGAATACCACG-3′ | 5′-FAM |
| | 19 E | FAM_E2 | | labeled |
| | gene | | | |
| 43 | Covid- | LB_ | 5′-GTTAACGTGAGTCTTG-3′ | 5′-FAM |
| | 19 E | FAM_E2 | | labeled |
| | gene | | | |

E3 Primer set

| | | | | |
|---|---|---|---|---|
| 44 | Covid- | F3_E3 | 5′-TCATTCGTTTCGGAAGAGA-3′ | N/A |
| | 19 E | | | |
| | gene | | | |
| 45 | Covid- | B3_E3 | 5′-AGGAACTCTAGAAGAATTCAGAT-3′ | N/A |
| | 19 E | | | |
| | gene | | | |
| 46 | Covid- | FIP_E3 | 5′- | N/A |
| | 19 E | | TGTAACTAGCAAGAATACCACGAAACAGGTAC | |
| | gene | | GTTAATAGTTAATAGCG-3′ | |
| 47 | Covid- | BIP_E3 | 5′- | N/A |
| | 19 E | | GCTTCGATTGTGTGCGTACTCGAGAGTAAACGT | |
| | gene | | AAAAAGAAGG-3′ | |
| 48 | Covid- | LB_E3 | 5′-GCTGCAATATTGTTAACGTGAGTC-3′ | N/A |
| | 19 E | | | |
| | gene | | | |

TABLE 18

| | Species | Target | |
|---|---|---|---|
| Seq ID No | origin | region | Sequence |
| 49 | SARS- | E gene | ATGTACTCATTCGTTTCGGAAGAGACAGG |
| | CoV-2 | | TACGTTAATAGTTAATAGCGTACTTCTTT |
| | | | TTCTTGCTTTCGTGGTATTCTTGCTAGTTA |
| | | | CACTAGCCATCCTTACTGCGCTTCGATTG |
| | | | TGTGCGTACTGCTGCAATATTGTTAACGT |
| | | | GAGTCTTGTAAAACCTTCTTTTTACGTTTA |
| | | | CTCTCGTGTTAAAAATCTGAATTCTTCTA |
| | | | GAGTTCCTGATCTTCTGGTCTAA |

TABLE 19
E gene LAMP optimization

| | | Supplier/ | Mixed | Final |
|---|---|---|---|---|
| | | Manufac- | volume | concentration |
| No. | Reagents | turer | (if any) | per reaction |
| 1 | Mastermix | NEB | 12.5 μL | 1x |
| | (WARMSTART ® | | (50% total | |
| | LAMP Kit | | volume) | |
| | (DNA&RNA)) | | | |
| 2 | LAMP Fluorescence | NEB | 0.5 μL | 1x |
| | Dye (50X) | | (2% total | |
| | | | volume) | |
| 3 | Primer mix | Synbio | 2.5 μL | 1x |
| | (10x stock) | Technologies | (10% total | |
| | | | volume) | |

E1 primer set

| | | | | |
|---|---|---|---|---|
| A | Primer No. 26 | N/A | 0.12 | μM |
| B | Primer No. 27 | | 0.12 | μM |
| C | Primer No. 28 | | 1.6 | μM |
| D | Primer No. 29 | | 1.6 | μM |
| E | Primer No. 30 | | 0.4 | μM |
| F | Primer No. 31 | | 0.4 | μM |

E2 primer set

| | | | | |
|---|---|---|---|---|
| A | Primer No. 32 | N/A | 0.12 | μM |
| B | Primer No. 33 | | 0.12 | μM |
| C | Primer No. 34 | | 1.6 | μM |
| D | Primer No. 35 | | 1.6 | μM |
| E | Primer No. 36 | | 0.4 | μM |
| F | Primer No. 37 | | 0.4 | μM |

E3 primer set

| 3 | Primer mix | Synbio | 2.5 μL | 1x | |
|---|---|---|---|---|---|
| | (10x stock) | Technologies | (10% total | | |
| | | | volume) | | |
| A | Primer No. 44 | | N/A | 0.12 | μM |
| B | Primer No. 45 | | | 0.12 | μM |
| C | Primer No. 46 | | | 1.6 | μM |
| D | Primer No. 47 | | | 1.6 | μM |
| E | Primer No. 48 | | | 0.4 | μM |

| | | | | |
|---|---|---|---|---|
| 4 | Sample | IDT | 5 μL | 1:100k to 1:10M |
| | | | (25% total | dilution |
| | | | volume) | |
| 5 | dH₂O | — | To 25 μL | — |

Temperature screening for all 3 primer sets were performed based on Table 19 to check the suitable reaction temperature. LAMP is operable in a wide range of temperatures ranging from 60 to 69° C. with 65° C. as optimal temperature, but different sets of primers might have different optimal temperatures. (Liu et al., “Establishment of an accurate and fast detection method using molecular beacons in loop-mediated isothermal amplification assay”, Sci Rep, 7, 40125 (2017)). Thus, the primer sets were screened in 4 temperature points from 65 to 59° C. with the same amount of sample. Samples used were self-designed plasmid containing E gene sequence and covering all 3 primer set target regions, including the E gene upstream target of E1 primer set.

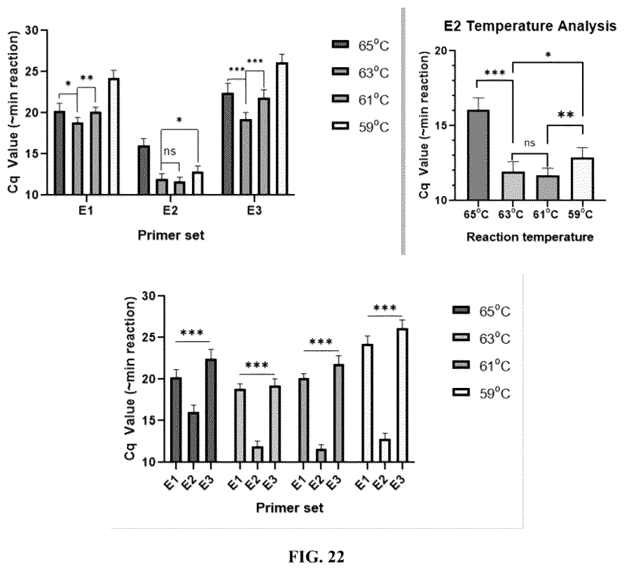

The result of comparing 3 primer sets for E gene is displayed in FIG. 22. The Cq values of the reaction demonstrate the speed of the reaction, and more efficient primer set will give lower Cq value. In all primer sets, LAMP reaction in temperature 63° C. resulted in the lowest Cq value, which demonstrated that this temperature gave the most efficient amplification throughout all primer sets, indicating the temperature is the best for the reaction. Statistical tests comparing 63° C. with 2 other temperatures with similar Cq values confirmed that reaction in 63° C. provides the best result in all primer sets. However, E2 primer set showed that 61° C. has similar efficiency with 63° C., indicating that this primer set works efficiently in the 61-63° C. temperature range.

Next, the efficiency of each LAMP primer set was compared. Comparing primer sets on each temperature displayed significant difference between E2 and the remaining primer sets. Across all temperatures, E2 showed better amplification efficiency. Overall, E2 primer set reached the positive threshold around 5-10 minutes faster than E1 or E3 primer sets, making it the most efficient primer set for E gene detection. Thus, E2 primer set was selected for further optimization.

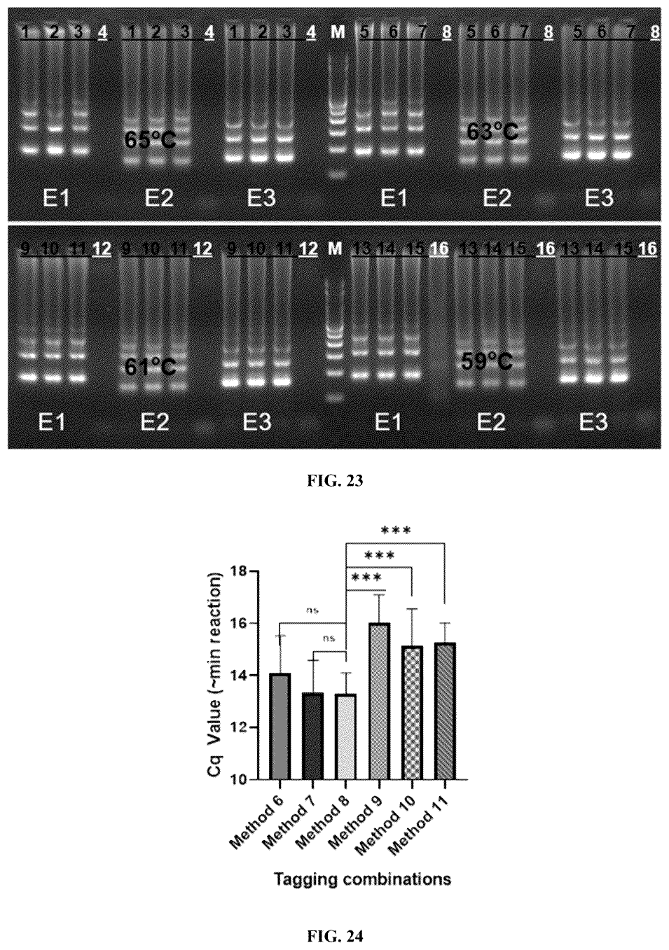

Gel electrophoresis of the screening results showed all primer set samples in 4 temperatures showed the characteristic LAMP “multiple bands” pattern (FIG. 23). At lower temperature, false positive results may be detected (E1 NTC/no. 16). The lower temperature reaction increases the chance of false positive due to formation of secondary structures, primer dimers, or non-specific amplification. (Ozay and McCalla, “A review of reaction enhancement strategies for isothermal nucleic acid amplification reactions”, Sensors and Actuators Reports, Volume 3, November 2021, 100033). Thus, a higher temperature is preferred when it is possible, and for further testing, 63° C. was selected for reaction temperature to minimize false positive occurrence.

Detection of LAMP results in LFA are achieved by addition of hapten tags into the reaction, allowing interaction of amplicons containing tagged primers or probes with antibodies in the paper LFA. Previous reports on LAMP-LFA describes various tagging mechanisms, ranging from addition of tagged probe (Jawla et al., “Paper-based loop-mediated isothermal amplification and lateral flow (LAMP-LF) assay for identification of tissues of cattle origin”, Analytica Chimica Acta, Volume 1150, 15 Mar. 2021, 338220; Jawla et al., “On-site paper-based Loop-Mediated Isothermal Amplification coupled Lateral Flow Assay for pig tissue identification targeting mitochondrial CO I gene”, Journal of Food Composition and Analysis, Volume 102, September 2021, 104036), inclusion of tagged dUTP nucleotides (Agarwal et al., “Lateral flow-based nucleic acid detection of SARS-COV-2 using enzymatic incorporation of biotin-labeled dUTP for POCT use”, Anal Bioanal Chem, 414, 3177-3186 (2022)) or tagging some of the primers (Kim and Oh, “Development of a filtration-based LAMP-LFA method as sensitive and rapid detection of E. coli O157:H7”, J Food Sci Technol, 56, 2576-2583 (2019); Zhang et al., “Rapid One-Pot Detection of SARS-COV-2 Based on a Lateral Flow Assay in Clinical Samples”, Anal. Chem., 2021, 93, 7, 3325-3330 Feb. 11, 2021; Khangembam ct al., “Point of care colourimetric and lateral flow LAMP assay for the detection of Haemonchus contortus in ruminant faecal samples”, Parasite, 2021; 28:82; Anna Zasada et al., “Detection of SARS-COV-2 Using Reverse Transcription Helicase Dependent Amplification and Reverse Transcription Loop-Mediated Amplification Combined with Lateral Flow Assay”, Biomedicines, 2022, 10(9), 2329). Incorporating the hapten tag into the primers is the most popular method reported, but there is no clear analysis on the combination of the primers used for tagging. Therefore, we devised a method to screen the best primer tagging combination through development of tagging matrix to cover the possible primer-hapten combinations.

Through the proposed screening method, multiple combinations of hapten-primer tagging are tested to get the most efficient hapten-primer combinations. Extensive testing to screen the hapten tag and primer mixture was carried out to find the best pairing for this primer set. Four out of six primers in the LAMP set were included in the screening, excluding the forward and backward outer primers (F3 and B3). Three hapten tags (Bio, FAM and DIG) were selected to tag four possible LAMP primers, resulting in 36 possible primer combinations to screen, assuming the reverse pairing (e.g. FIP-Bio with LF-FAM and FIP-FAM with LF-Bio) is considered the same. From literature review (Basing et al., “A Loop-Mediated Isothermal Amplification Assay for the Detection of Treponema pallidum subsp. pertenue”, Am J Trop Med Hyg., July 2020; 103(1): 253-259; Ahn et al., “Zika virus lateral flow assays using reverse transcription-loop-mediated isothermal amplification”, RSC Advances, 2021, 11, 17800-17808; Lce et al., “A Lateral Flow Assay for Nucleic Acid Detection Based on Rolling Circle Amplification Using Capture Ligand-Modified Oligonucleotides”, BioChip Journal, 2022, 16, 441-450; Safenkova et al., “Key significance of DNA-target size in lateral flow assay coupled with recombinase polymerase amplification”, Analytica Chimica Acta, 15 Mar. 2020, 109-118; Rezaei et al., “A Portable RT-LAMP/CRISPR Machine for Rapid COVID-19 Screening”, Biosensors, 2021, 11(10), 369; Jang et al., “Rapid COVID-19 Molecular Diagnostic System Using Virus Enrichment Platform”, Biosensors, 2021, 11, 373), it is found that the Bio-FAM combination is more widely used than the Bio-DIG or FAM-DIG, thus the screening on Bio-FAM combination is the first priority in this example. The final candidates for screening were 6 combinations, henceforth identified as Method 6 to Method 11 (or M6 to M11, respectively) as displayed in Table 20.

TABLE 20
Primer tagging matrix for screening.
Primer tagging combination

| | | Tag 1-FAM | | | |
|---|---|---|---|---|---|
| | | FIP | BIP | LB | LF |
| Tag 2-Biotin | FIP | / | Method 11 | Method 8 | Method 7 |
| | BIP | Method 11 | / | Method 6 | Method 9 |
| | LB | Method 8 | Method 6 | / | Method 10 |
| | LF | Method 7 | Method 9 | Method 10 | / |

All 6 tagging combinations were tested simultaneously with E plasmid purchased from IDT to have better assessment on the sample copy number. From the result in Table 21 and FIG. 24, every tagging combination has different Cq values, indicating the choice of primers for tagging has an effect on the amplification. The lowest Cq, thus the most efficient amplification, is achieved by M8 combination, or tagging of FIP and LB. M7 has nearly similar efficiency to that of M8; M6 have slightly higher Cq value; and the rest having less efficient amplification. Statistical test (t-test) comparing M8 with other methods showed that M6 and M7 are not statistically different, while the remaining methods are significantly different. From this primer set, the best combination should involve 1 FIP/BIP primer with 1 LF/LB primer. Combination of both FIP-BIP (M11) or LF-LB (M10) would yield less efficient amplification.

TABLE 21

| Primer-Tag combinations | Average Cq | StDev |
|---|---|---|
| Method 6 | 14.08 | 1.45 |
| Method 7 | 13.33 | 1.26 |
| Method 8 | 13.28 | 0.83 |
| Method 9 | 16.04 | 1.06 |
| Method 10 | 15.12 | 1.44 |
| Method 11 | 15.25 | 0.77 |

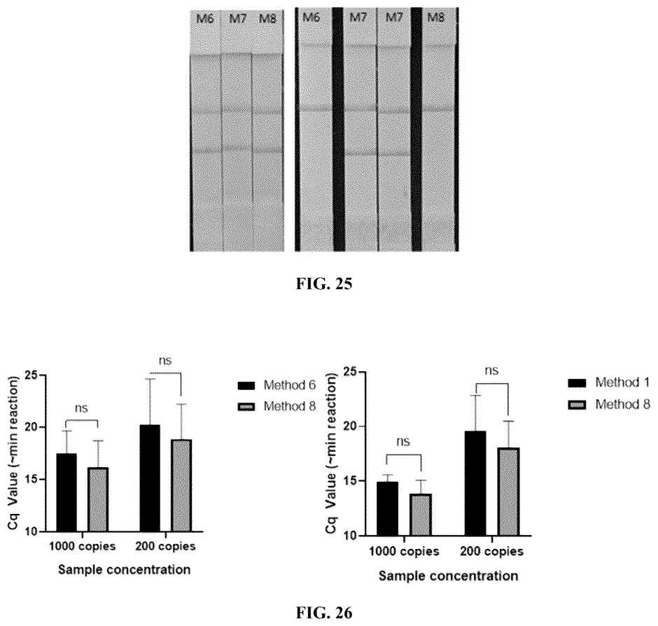

After the 6 tagging combination screening, the methods with better efficiency (M6-8) were tested with LFA to check the tagging performance. Positive samples amplified by primers M6-8 were run in LFA, giving positive results (formation of 2 bands) on all combinations (FIG. 25). However, the LFA test on NTC gives positive band, in 2 different NTCs done in independent replicates. The false positive shows that the M7 primer tagging combination (FIP and LF) has potential for self-reacting and causes LFA to be positive despite no amplification occurring. In contrast, NTC of M6 and M8 are negative, indicating that the primer tags do not self-react in the absence of target sequence.

The LFA test result shows that M6 and M8 are the suitable candidates for LFA adaptation. Further test was performed to check the best performing tag combination out of these 2. Both primer sets were tested on 2 different concentrations of sample, 1000 and 200 copies/reaction. In both concentrations, the Cq value of M8 was overall lower than M6, but the statistical test results show no statistical difference (FIG. 26). The results confirm that M8 is preferable combination due to its better Cq value, but M6 is an alternative for tagging with similar performance. Furthermore, a test with standard primer set without tag (M1) shows that Cq values of M8 had no significant difference with M1, indicating the addition of biotin and FAM into the primers does not affect the primer performance (FIG. 26).

TABLE 22
E gene LAMP amplification components

| | | | | |
|---|---|---|---|---|
| | | Supplier/ | Mixed | Final |
| | | Manufac- | volume | concentration |
| No. | Reagents | turer | (if any) | per reaction |

Fluorescence-LFA based test

| 1 | Mastermix | NEB | 12.5 μL | 1x | |
|---|---|---|---|---|---|
| | (WARMSTART ® | | (50% total | | |
| | LAMP Kit | | volume) | | |
| | (DNA&RNA)) | | | | |
| 2 | LAMP Fluorescence | NEB | 0.5 μL | 1x | |
| | Dye (50X) | | (2% total | | |
| | | | volume) | | |
| 3 | Primer mix (10x stock) | Synbio | 2.5 μL | 1x | |
| | | Technol- | (10% total | | |
| | | ogies | volume) | | |
| A | Primer No. 32 | | N/A | 0.12 | μM |
| B | Primer No. 33 | | | 0.12 | μM |
| C | Primer No. 38 | | | 1.6 | μM |
| D | Primer No. 35 | | | 1.6 | μM |
| E | Primer No. 36 | | | 0.4 | μM |
| F | Primer No. 43 | | | 0.4 | μM |

| | | | | |
|---|---|---|---|---|
| 4 | Sample | IDT | 5 μL | 1000 to 200 |
| | | | (25% total | copies/reaction |

volume)

| | | | | |
|---|---|---|---|---|
| 5 | dH₂O | — | To 25 μL | — |

Real-time LFA (Colorimetric-Fluorimetric-LFA)

| 1 | Mastermix | NEB | 12.5 μL | 1x | |
|---|---|---|---|---|---|
| | (WARMSTART ® | | (50% total | | |
| | Colorimetric | | volume) | | |
| | LAMP 2X | | | | |
| | Master Mix | | | | |
| | (DNA&RNA)) | | | | |
| 2 | LAMP Fluorescence | NEB | 0.5 μL | 1x | |
| | Dye (50X) | | (2% total | | |
| | | | volume) | | |
| 3 | Primer mix | Synbio | 2.5 μL | 1x | |
| | (10x stock) | Technol- | (10% total | | |
| | | ogies | volume) | | |
| a | Primer No. 32 | | N/A | 0.12 | μM |
| b | Primer No. 33 | | | 0.12 | μM |
| c | Primer No. 38 | | | 1.6 | μM |
| d | Primer No. 35 | | | 1.6 | μM |
| e | Primer No. 36 | | | 0.4 | μM |
| f | Primer No. 43 | | | 0.4 | μM |

| | | | | |
|---|---|---|---|---|
| 4 | Sample | IDT | 5 μL | 20000 to 200 |
| | | | (25% total | copies/reaction |

volume)

| | | | |
|---|---|---|---|
| 5 | dH₂O | To 25 μL | — |

Dry reactions - Real-time LFA

| 1 | Haigene Bst 4.0 | Haigene | N/A | 1 | bead |
|---|---|---|---|---|---|
| | red/pH beads | | | | |
| | (A3828-01R) | | | | |
| 2 | LAMP Fluorescence | NEB | 0.5 μL | 1x | |
| | Dye (50X) | | (2% total | | |
| | | | volume) | | |
| 3 | Primer mix | Synbio | 2 μL | 1x | |
| | (10x stock) | Technol- | (10% total | | |
| | | ogies | volume) | | |
| a | Primer No. 32 | | N/A | 0.12 | μM |
| b | Primer No. 33 | | | 0.12 | μM |
| c | Primer No. 38 | | | 1.6 | μM |
| d | Primer No. 35 | | | 1.6 | μM |
| e | Primer No. 36 | | | 0.4 | μM |
| f | Primer No. 43 | | | 0.4 | μM |

| | | | | |
|---|---|---|---|---|
| 4 | Sample | IDT | 5 μL | 500 |
| | | | (25% total | copies/reaction |

volume)

| | | | | |
|---|---|---|---|---|
| 5 | dH₂O | — | To 25 μL | — |

The specificity of the selected E2 primer set was further tested against several bacterial DNA samples and human DNA extracted from HeLa and 293T cell line by methods described in Table 22 (Fluorescence based test). All human and bacterial DNA samples were used in 25 ng concentration and performed in triplicates. One positive control with 1:100 k dilution was included for reaction control.

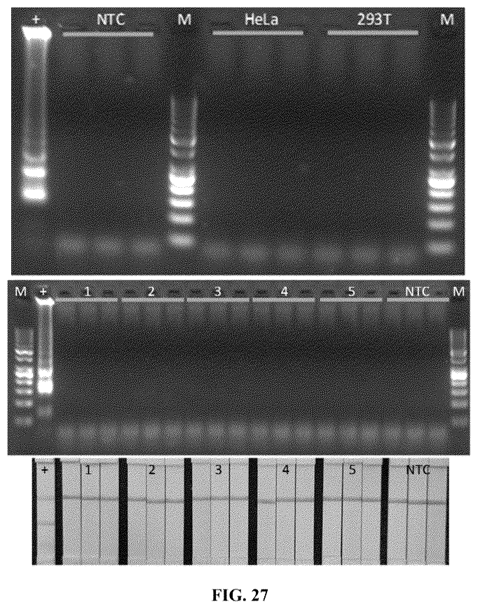

Table 23 showed results of specificity tests on DNA extracted from HeLa and 293T cell lines to replicate human DNA showed no amplification result. Tests on bacterial DNA samples resulted in no amplification in all samples. Furthermore, as HeLa cells possess a sequence of HPV, no amplification in DNA samples demonstrates the primer set remains specific to SARS-CoV-2 E gene. Reactions using various bacterial DNA (FIG. 27) showed no false positive result.

TABLE 23

| | | Positive | Average Cq |
|---|---|---|---|
| Samples | No | rate | value |
| 1:100k dilution positive control plasmid | + | 2/2 | 11.94 |
| HeLa cell line DNA | HeLa | 0/3 | N/A |
| 293T cell line DNA | 293T | 0/3 | N/A |
| Klebsiella pneumoniae (ATCC78578) | 1 | 0/3 | N/A |
| Klebsiella pneumoniae (ATCC13883) | 2 | 0/3 | N/A |
| Acinetobacter (ATCC19606) | 3 | 0/3 | N/A |
| Pseudomonas protegens | 4 | 0/3 | N/A |
| (ATCCBAA-477TM) | | | |
| E. coli (ATCC25922) | 5 | 0/3 | N/A |

### Real Time LFA

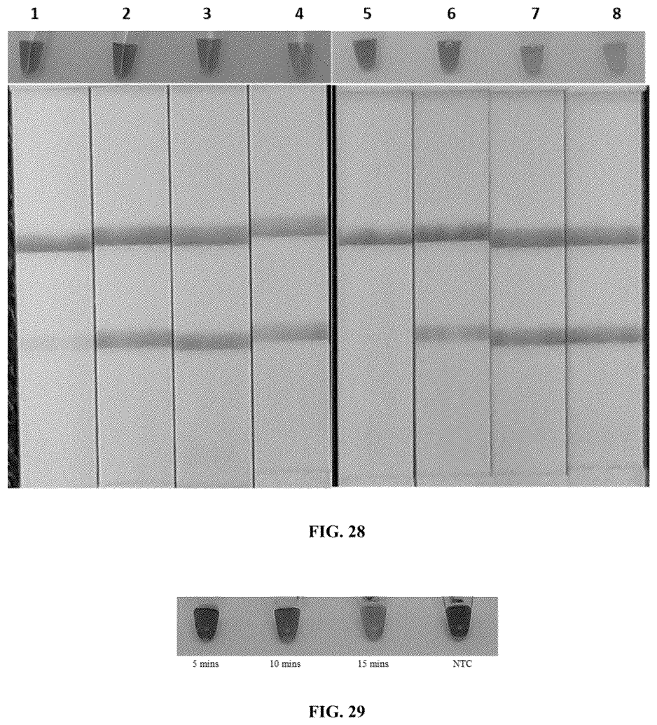

The developed M8 primer set was tested for real-time LFA, a test combining colorimetric-fluorimetric real time testing with LFA confirmatory analysis. The components of the test are described in Table 22 with reaction settings in Table 8 above, and the test was performed by incubating samples with 2 different concentrations. A high amount of plasmid (20000 cps/reaction) and low amount of plasmid (200 cps/reaction) were tested simultaneously in fluorimetric, colorimetric and LFA. Each sample was prepared in pH based colorimetric master mix and placed in thermocycler, then retrieved after a certain duration and kept in ice to terminate the reaction. In the same time, the reaction was run in Bio-Rad CFX Connect to measure the endpoint based on the fluorimetric method (Table 24). LFA analysis on the samples stopped at different time points showed the reaction had reached a positive threshold before stipulated 30 min reaction duration (FIG. 28) Amplicons from 20000 cps/reaction samples gave a light positive band in 10 min incubation, close to the Cq value from the fluorimetric reading, which corresponds to time it became positive (Cq 12.06 or around 12 minutes). In lower concentration 200 copies/reaction, LFA started turn positive after 15 mins of incubation, close to the fluorimetric result of Cq 13.57 (around 13.5 minutes to reach positive). Noticeably, the color change on this system lags the LFA positive result. Sample 6 was red in color despite the positive LFA results, while sample 2 and 7 were slightly orange and both are positive in LFA. Thus, a change in color, even to slight orange, indicated that the reaction can be stopped and checked in LFA, reducing the time by 10 to 15 minutes if depending solely on color change.

TABLE 24

| Primer-Tag | Average Cq | Sample | Incubation | LFA |
|---|---|---|---|---|
| combinations | Value | No. | duration | Result |
| 20000 cps/reaction | 12.06 | 1 | 10 min | + |
| | | 2 | 15 min | + |
| | | 3 | 20 min | + |
| | | 4 | 30 min | + |
| 200 cps/reaction | 13.57 | 5 | 10 min | − |
| | | 6 | 15 min | + |
| | | 7 | 20 min | + |
| | | 8 | 30 min | + |

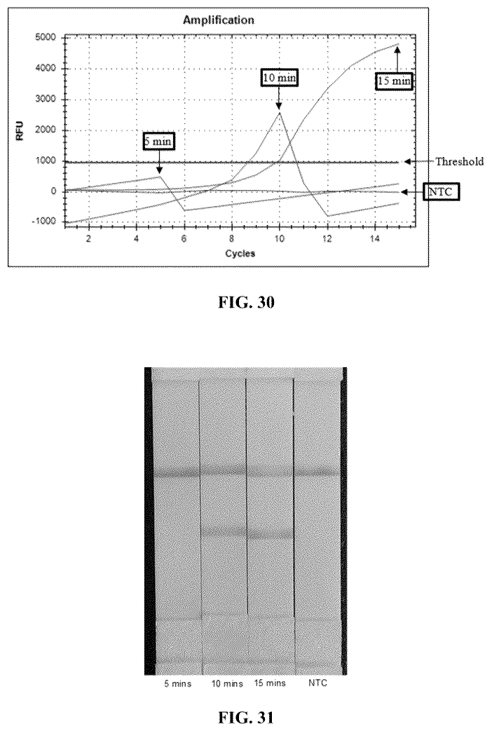

Next, the reaction of 500 copies/reaction samples stopped in 3 different incubation time intervals to check the correlation of fluorescence peak with the LFA positive rate. FIGS. 29-31 displayed the results of the real-time LFA test. As seen in FIGS. 29-31, 5 minutes of incubation did not change the color and did not obtain any peak in the fluorescence graph. Through LFA analysis (FIG. 31), it is confirmed that there was no amplicon detected yet. At the 10th minute of the incubation, the color started to change from red into slightly orange (FIG. 29), and the fluorescence peak also spiked to form a peak over the threshold limit (FIG. 30), indicating a positive result. In the LFA analysis (FIG. 31), the sample showed positive result (2 bands), which cut the incubation time by 20 mins (from 30 to 10 mins). Sample taken in 15 mins also showed all positive in 3 different analyses.

From the results of this example, the combination of fluori-colorimetric and LFA analyses has successfully displayed the integration of the real-time LAMP analysis (fluorimetric and colorimetric) with the highly specific and sensitive LFA analysis. The real-time LFA combines the positive aspect of the constituting methods (faster, specific results) and eliminating the negative aspects of the methods (prone to false positive or longer duration due to end-point result needed for LFA analysis). Moreover, the series of tests have demonstrated that the present method according to certain embodiments is an efficient screening method for determining the best hapten tag-primer pairings, and showed the compatibility of the pairings with the real-time LFA method.

Although the invention has been described in terms of certain embodiments, other embodiments apparent to those of ordinary skill in the art are also within the scope of this invention. Accordingly, the scope of the invention is intended to be defined only by the claims which follow.

### INDUSTRIAL APPLICABILITY

The present invention significantly shortens the conventional LAMP-based detection method by integrating multiple result interpretation platform including the LFA and colorimetric/fluorimetric assays. Not just improving the detection efficiency, validity of positive results is assured as LFA imparts high specificity and the incorporation thereof intervenes the LAMP reaction when sufficient copies of positive amplicons are present, in order to reduce the likelihood of having false positives arising from non-specific amplification reaction during the remainder of the LAMP reaction. Therefore, the proposed integrated immunochromatographic assay into the LAMP-based detection system can bring grounds for developing into a small-sized or even portable diagnostic tool for rapid pathogen detection with high specificity. The present invention can also be developed into a high throughput pathogen detection system in a reasonable and affordable cost.

What is claimed is:

1. A pathogen detection system comprising a reaction mixture for reacting with an analyte, the reaction mixture comprising:
a colorimetric indicator responsive to a change brought by a potential positive nucleic acid amplification in the reaction mixture;
a fluorescent indicator responsive to a presence of amplicons from an amplification reaction of each of target sequences in the analyte;
a plurality of primers complementary to one or more independent regions in each of the target sequences to be amplified; and
at least one enzyme for initiating the amplification reaction of the target sequences;
a master mix for said amplification reaction,
the pathogen detection system further comprising a concurrent immunochromatographic assay comprising an antibody specific to hapten-tag identifiers of the amplicons representing one of the target sequences.

2. The pathogen detection system of claim 1, wherein the amplification reaction of the target sequences comprises loop-mediated isothermal amplification (LAMP).

3. The pathogen detection system of claim 1, wherein some of the plurality of primers except a forward outer primer (F3) and a reverse outer primer (B3) are conjugated with at least two different kinds of tags such that the at least two kinds of tags are contained in corresponding amplicons as the identifier which are specifically bound with said antibody in the concurrent immunochromatographic assay.

4. The pathogen detection system of claim 3, wherein the two different kinds of tags are two different hapten tags selected from any two of various hapten tags comprising biotin, 6-carboxyfluorescein (FAM), fluorescein isothiocyanate (FITC), digoxigenin (DIG), tetramethyl rhodamine (TAMRA), dinitrophenyl and sulforhodamine (Texas Red) that are conjugated to 5′-end of some of the plurality of primers, and wherein the plurality of primers conjugated at the 5′-end thereof with said two different hapten tags comprises a reverse inner primer (BIP) or a forward inner primer (FIP) and a loop backward (LB) primer.

5. The pathogen detection system of claim 1, wherein the fluorescent indicator is one or more fluorescent dyes comprising SYBR green, SYTO-82, and SYTO-84 that intercalates with the amplicons of one of the target sequences to emit fluorescence signal.

6. The pathogen detection system of claim 1, wherein the target sequence to be amplified into the amplicon with which said antibody specifically binds to the hapten-tag identifier thereof or the fluorescent indicator intercalates is a target sequence of a pathogen in the analyte.

7. The pathogen detection system of claim 6, wherein the pathogen comprises various kinds of biological materials comprising viruses, bacteria, fungi, and other pathogens.

8. The pathogen detection system of claim 6, wherein the analyte comprises nucleic acid samples from mammalian cells.

9. The pathogen detection system of claim 1, wherein the colorimetric indicator is a colorimetric dye selected from various dyes which detect the changes in the reaction that pinpoints an amplification of the target nucleic acid comprising halochromic dye or metallochromic dye.

10. The pathogen detection system of claim 9, wherein the halochromic dye comprises phenol red, methyl red, bromothymol blue, phenolphthalein, and triarylmethane; the metallochromic dye comprises hydroxynaphtol blue.

11. The pathogen detection system of claim 1, wherein the sample comprises DNA molecules, RNA molecules, or a biological agent containing any of the DNA and RNA molecules.

12. The pathogen detection system of claim 1, wherein the enzyme for initiating the amplification reaction of the target sequences comprises DNA polymerase with strand displacement and/or reverse transcription activities and reverse transcriptase.

13. A method for improving detection efficiency and validity of a molecular diagnostic assay on a target pathogen gene in an analyte, comprising:
providing the reaction mixture of the pathogen detection system according to claim 1;
initiating an amplification of target nucleic acid by enzymes in the reaction mixture in a platform capable of generating constant heat;
performing colorimetric and/or fluorimetric analyses during said amplification by observing any change in color and/or spiked emission of fluorescence signal;
if any color change or spiked emission of fluorescence being observed, stopping said platform and subjecting reaction product obtained instantly after said stopping to an immunochromatographic assay;
validating the result of said molecular diagnostic assay by a corresponding visible signal obtained from the immunochromatographic assay and comparing said visible signal with a gel electrophoresis analytical data of said reaction product; and
if said visible signal giving a coherent positive result representing a presence of the target pathogen gene in the analyte, a thermocycle at which said reaction product being validated as positive by said immunochromatographic assay representing an end of the molecular diagnostic assay.

14. The method of claim 13, wherein said amplification of target nucleic acid in the reaction mixture directly comprises loop-mediated isothermal amplification (LAMP) reaction.

15. The method of claim 13, wherein said platform comprises a thermocycler, water bath or heat block.

16. The method of claim 13, wherein the color change is due to a halochromic change or metallochromic change during said amplification of the target nucleic acid in the reaction mixture, which is indicative of a presence of amplicons from said reaction.

17. The method of claim 13, wherein the spiked emission of fluorescence signal emission is indicative of a presence of amplicons from an amplification reaction.

18. The method of claim 13, wherein the immunochromatographic assay is performed in a lateral flow device.

19. A method for identifying a target gene in a biological sample with a molecular diagnostic assay compatible with a plurality of detection methods that include fluorimetric, colorimetric, immunochromatographic assay based on lateral flow assay and gel electrophoresis, the method comprising using the pathogen detection system of claim 1.

20. The method of claim 19, wherein either or both of colorimetric and fluorimetric detection methods is or are incorporated with the lateral flow assay detection method to perform the molecular diagnostic assay selected from a real-time LAMP.

21. A kit for a one-pot pathogen detection in an analyte comprising the pathogen detection system according to claim 1 either formulated in wet or dry chemistry form.


SEQUENCE LISTING (111 sequences)

SEQ ID NO: 1 - DNA, 39 bp, synthetic construct
  source 1..39: mol_type = other DNA
gaagcgtaagttcccggaggcaacgaatacagaagccgt

SEQ ID NO: 2 - DNA, 19 bp, synthetic construct
  source 1..19: mol_type = other DNA
accatccaatggaaagtgt

SEQ ID NO: 3 - DNA, 49 bp, synthetic construct
  source 1..49: mol_type = other DNA
aagactaatacacaatgtacaccgtcctgtaaatgattattgtgagatg

SEQ ID NO: 4 - DNA, 21 bp, synthetic construct
  source 1..21: mol_type = other DNA
ttccgttacaacttagacaag

SEQ ID NO: 5 - DNA, 21 bp, synthetic construct
  source 1..21: mol_type = other DNA
acacgatagacaacatagatt

SEQ ID NO: 6 - DNA, 18 bp, synthetic construct
  source 1..18: mol_type = other DNA
  misc_feature 1: note = 5'-FAM labeled
gtggttcggataccttta

SEQ ID NO: 7 - DNA, 39 bp, synthetic construct; identical to SEQ ID NO: 1
  source 1..39: mol_type = other DNA
  misc_feature 1: note = 5'-biotin labeled

SEQ ID NO: 8 - DNA, 49 bp, synthetic construct; identical to SEQ ID NO: 3
  source 1..49: mol_type = other DNA
  misc_feature 1: note = 5'-biotin labeled

SEQ ID NO: 9 - DNA, 18 bp, synthetic construct
  source 1..18: mol_type = other DNA
  misc_feature 1: note = 5'-biotin labeled
gtggttcggataccttta

SEQ ID NO: 10 - DNA, 39 bp, synthetic construct; identical to SEQ ID NO: 1
  source 1..39: mol_type = other DNA
  misc_feature 1: note = 5'-FAM labeled

SEQ ID NO: 11 - DNA, 21 bp, synthetic construct
  source 1..21: mol_type = other DNA
  misc_feature 1: note = 5'-FAM labeled
acacgatagacaacatagatt

SEQ ID NO: 12 - DNA, 18 bp, synthetic construct
  source 1..18: mol_type = other DNA
  misc_feature 1: note = 5'-FAM labeled
gtggttcggataccttta

SEQ ID NO: 13 - DNA, 50 bp, synthetic construct
  source 1..50: mol_type = other DNA
tggagtctgctaatctctgtaagattagagaactagagaataagttgacc

SEQ ID NO: 14 - DNA, 20 bp, synthetic construct
  source 1..20: mol_type = other DNA
cacaagaagttgatgcactg

SEQ ID NO: 15 - DNA, 39 bp, synthetic construct
  source 1..39: mol_type = other DNA
tgagtgaatgccgtggaaatgcgcagtcgttcaactgta

SEQ ID NO: 16 - DNA, 23 bp, synthetic construct
  source 1..23: mol_type = other DNA
cagcattgatttcattattacgt

SEQ ID NO: 17 - DNA, 16 bp, synthetic construct
  source 1..16: mol_type = other DNA
cgctctcgatgcagtc

SEQ ID NO: 18 - DNA, 25 bp, synthetic construct
  source 1..25: mol_type = other DNA
cagagattacaatctagaatctcag

SEQ ID NO: 19 - DNA, 39 bp, synthetic construct; identical to SEQ ID NO: 15
  source 1..39: mol_type = other DNA
  misc_feature 1: note = 5'-biotin labeled

SEQ ID NO: 20 - DNA, 25 bp, synthetic construct
  source 1..25: mol_type = other DNA
  misc_feature 1: note = 5'-FAM labeled
cagagattacaatctagaatctcag

SEQ ID NO: 21 - DNA, 660 bp, Monkeypox virus
  source 1..660: mol_type = other DNA
gacacacaagaagttgatgcactgcgttcgcgtattagagaactagagaataagttgaccgactgcatcgagagcggaggaggaaatcttacagagattagcagactccaatctagaatctcagatcttgaaagacaactgagtgaatgccgtggaaatgctacagagattacaatctagaatctcagatcttgaaatacagttgaacgactgcggacgtaataatgaaatcaatgctgatatggaaaagagatagaatcatggatcttgatagacatcttaacgattgtaaaaacggtaacggagcatcttctgaggaggtaaataggctaaagactagaatcagggatcttgaacgatcgctagagatcttctcaaaggatgaatcagaactctattcggcatataaaactgaactcggtaatatcagcacttaataataaacggaagagatatagcaccacatgcaccatccaatggaaagtgtaaagacaacgaatacagaagccgtaatctatgttgtctatcgtgtcctccgggaacttacgcttccagattatgtgatagcaagactaatacacaatgtacaccgtgtggttcggatacctttacatctcacaataatcatttacaggcttgtctaagttgtaacggaagatg

SEQ ID NO: 22 - DNA, 519 bp, Monkeypox virus
  source 1..519: mol_type = other DNA
ttaagccgctagaagttttccgtttgatataggatgtggacatttaacaatctgacacgtgggtggattggaccattctcctcctgaacacatgacaccagagttaccaatcaacgaatatccactattgcaactataagttacaatgctcccatcgatataaaaatcctcgtatccgttatgtcttccgttggatatagatggaggtgattggcatttaacagattcgcaaataggtgcctcaggattccataccatagatccagtagatcctaattcacaatacgatttagattcaccgatcaaatgatatccgctattacaagagtacgttatactagagccaaagtctactccgccaatatcaagttggccattatcgatatctcgaggcgatgggcatctccgtttaatacattgattaaagagtgtccatccggtaccggtacatttagcatatatgggtcccattttttgctttctgtatccaggtagacatagatattctatagtgtctcc

SEQ ID NO: 23 - DNA, 26 bp, synthetic construct
  source 1..26: mol_type = other DNA
ggaaaatgtaaagacaacgaatacag

SEQ ID NO: 24 - DNA, 24 bp, synthetic construct
  source 1..24: mol_type = other DNA
gctatcacataatctggaagcgta

SEQ ID NO: 25 - DNA, 30 bp, synthetic construct
  source 1..30: mol_type = other DNA
  misc_feature 1: note = 5'-FAM labeled
  misc_feature 30: note = 3'-BHQ quencher
aagccgtaatctatgttgtctatcgtgtcc

SEQ ID NO: 26 - DNA, 18 bp, synthetic construct
  source 1..18: mol_type = other DNA
ccgacgacgactactagc

SEQ ID NO: 27 - DNA, 23 bp, synthetic construct
  source 1..23: mol_type = other DNA
agagtaaacgtaaaaagaaggtt

SEQ ID NO: 28 - DNA, 41 bp, synthetic construct
  source 1..41: mol_type = other DNA
acctgtctcttccgaaacgaatttgtaagcacaagctgatg

SEQ ID NO: 29 - DNA, 41 bp, synthetic construct
  source 1..41: mol_type = other DNA
ctagccatccttactgcgctactcacgttaacaatattgca

SEQ ID NO: 30 - DNA, 19 bp, synthetic construct
  source 1..19: mol_type = other DNA
tcgattgtgtgcgtactgc

SEQ ID NO: 31 - DNA, 19 bp, synthetic construct
  source 1..19: mol_type = other DNA
tgagtacataagttcgtac

SEQ ID NO: 32 - DNA, 19 bp, synthetic construct
  source 1..19: mol_type = other DNA
tttcggaagagacaggtac

SEQ ID NO: 33 - DNA, 22 bp, synthetic construct
  source 1..22: mol_type = other DNA
aggaactctagaagaattcaga

SEQ ID NO: 34 - DNA, 43 bp, synthetic construct
  source 1..43: mol_type = other DNA
cgcagtaaggatggctagtgtagcgtacttctttttcttgctt

SEQ ID NO: 35 - DNA, 43 bp, synthetic construct
  source 1..43: mol_type = other DNA
tcgattgtgtgcgtactgctgtttttaacacgagagtaaacgt

SEQ ID NO: 36 - DNA, 17 bp, synthetic construct
  source 1..17: mol_type = other DNA
ctagcaagaataccacg

SEQ ID NO: 37 - DNA, 16 bp, synthetic construct
  source 1..16: mol_type = other DNA
gttaacgtgagtcttg

SEQ ID NO: 38 - DNA, 43 bp, synthetic construct; identical to SEQ ID NO: 34
  source 1..43: mol_type = other DNA
  misc_feature 1: note = 5'-biotin labeled

SEQ ID NO: 39 - DNA, 43 bp, synthetic construct; identical to SEQ ID NO: 35
  source 1..43: mol_type = other DNA
  misc_feature 1: note = 5'-biotin labeled

SEQ ID NO: 40 - DNA, 16 bp, synthetic construct
  source 1..16: mol_type = other DNA
  misc_feature 1: note = 5'-biotin labeled
gttaacgtgagtcttg

SEQ ID NO: 41 - DNA, 43 bp, synthetic construct; identical to SEQ ID NO: 34
  source 1..43: mol_type = other DNA
  misc_feature 1: note = 5'-FAM labeled

SEQ ID NO: 42 - DNA, 17 bp, synthetic construct
  source 1..17: mol_type = other DNA
  misc_feature 1: note = 5'-FAM labeled
ctagcaagaataccacg

SEQ ID NO: 43 - DNA, 16 bp, synthetic construct
  source 1..16: mol_type = other DNA
  misc_feature 1: note = 5'-FAM labeled
gttaacgtgagtcttg

SEQ ID NO: 44 - DNA, 19 bp, synthetic construct
  source 1..19: mol_type = other DNA
tcattcgtttcggaagaga

SEQ ID NO: 45 - DNA, 23 bp, synthetic construct
  source 1..23: mol_type = other DNA
aggaactctagaagaattcagat

SEQ ID NO: 46 - DNA, 49 bp, synthetic construct
  source 1..49: mol_type = other DNA
tgtaactagcaagaataccacgaaacaggtacgttaatagttaatagcg

SEQ ID NO: 47 - DNA, 43 bp, synthetic construct
  source 1..43: mol_type = other DNA
gcttcgattgtgtgcgtactcgagagtaaacgtaaaaagaagg

SEQ ID NO: 48 - DNA, 24 bp, synthetic construct
  source 1..24: mol_type = other DNA
gctgcaatattgttaacgtgagtc

SEQ ID NO: 49 - RNA, 228 nt, Severe acute respiratory syndrome-related coronavirus
  source 1..228: mol_type = genomic RNA
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 50 - RNA, 188 nt, Monkeypox virus
  source 1..188: mol_type = genomic RNA
accatccaatggaaagtgtaaagacaacgaatacagaagccgtaatctatgttgtctatcgtgtcctccgggaacttacgcttccagattatgtgatagcaagactaatacacaatgtacaccgtgtggttcggatacctttacatctcacaataatcatttacaggcttgtctaagttgtaacggaa

SEQ ID NO: 51 - RNA, 188 nt, Monkeypox virus; identical to SEQ ID NO: 50
  source 1..188: mol_type = genomic RNA

SEQ ID NO: 52 - RNA, 188 nt, Monkeypox virus; identical to SEQ ID NO: 50
  source 1..188: mol_type = genomic RNA

SEQ ID NO: 53 - RNA, 188 nt, Monkeypox virus; identical to SEQ ID NO: 50
  source 1..188: mol_type = genomic RNA

SEQ ID NO: 54 - RNA, 188 nt, Monkeypox virus
  source 1..188: mol_type = genomic RNA
accatccaatggaaagtgtaaagacaacgaatacagaagccgtaatctatgttgtctatcgtgtcctccgggaacttacgctttcagattatgtgatagcaagactaatacacaatgtacaccgtgtggttcggatacctttacatctcacaataatcatttacaggcttgtctaagttgtaacggaa

SEQ ID NO: 55 - RNA, 188 nt, Monkeypox virus; identical to SEQ ID NO: 54
  source 1..188: mol_type = genomic RNA

SEQ ID NO: 56 - RNA, 188 nt, Monkeypox virus; identical to SEQ ID NO: 54
  source 1..188: mol_type = genomic RNA

SEQ ID NO: 57 - RNA, 188 nt, Monkeypox virus; identical to SEQ ID NO: 50
  source 1..188: mol_type = genomic RNA

SEQ ID NO: 58 - RNA, 188 nt, Monkeypox virus; identical to SEQ ID NO: 54
  source 1..188: mol_type = genomic RNA

SEQ ID NO: 59 - RNA, 188 nt, Monkeypox virus; identical to SEQ ID NO: 54
  source 1..188: mol_type = genomic RNA

SEQ ID NO: 60 - RNA, 188 nt, Monkeypox virus; identical to SEQ ID NO: 54
  source 1..188: mol_type = genomic RNA

SEQ ID NO: 61 - RNA, 188 nt, Monkeypox virus; identical to SEQ ID NO: 54
  source 1..188: mol_type = genomic RNA

SEQ ID NO: 62 - RNA, 188 nt, Monkeypox virus
  source 1..188: mol_type = genomic RNA
accatccaatggaaaatgtaaagacaacgaatacagaagccgtaatctatgttgtctatcgtgtcctccgggaacttacgcttccagattatgtgatagcaagactaatacacaatgtacgccgtgtggttcggatacctttacatctcacaataatcatttacaggcttgtctaagttgtaacggaa

SEQ ID NO: 63 - RNA, 188 nt, Variola virus
  source 1..188: mol_type = genomic RNA
accacccaatggaaagtgtaaagacaccgaatacaaacgccataatctgtgttgtttatcgtgtcctccgggaacatacgcttccagattatgtgatagcaagactaacacacaatgtacaccgtgtggttcgggtacctttacatctcgcaataatcatttacccgcttgtctaagttgtaacggaa

SEQ ID NO: 64 - RNA, 188 nt, Variola virus; identical to SEQ ID NO: 63
  source 1..188: mol_type = genomic RNA

SEQ ID NO: 65 - RNA, 188 nt, Camelpox virus
  source 1..188: mol_type = genomic RNA
accatccaatggaaagtgtaaagacaacgaatacaaacgccataatctgtgttgtttatcgtgtcctccgggaacatacgcttccagattatgcgatagcaagactaacacacaatgtacgccgtgtggttcgggtaccttcacatctcgcaataatcatttacccgcttgtctaagttgtaacggaa

SEQ ID NO: 66 - RNA, 188 nt, Camelpox virus
  source 1..188: mol_type = genomic RNA
accatccaatggaaagtgtaaagacaacgaatacaaacgccataatctgtgttgtttatcgtgtcctccgggaacatacgcttccagattatgcgatagcaagactaacacacaatgtacgccgtgtggttcgggtaccttcacatctcacaataatcatttacccgcttgtctaagttgtaacggaa

SEQ ID NO: 67 - RNA, 194 nt, Cowpox virus
  source 1..194: mol_type = genomic RNA
accatccaatggaaagtgtaaagacaacgaatacaaccgccataatctgtgttgtttatcgtgtcctccgggaacatacgcttccagattatgcgatagcaagactaacacaaacacacaatgtacgccgtgtggttcgggtacctttacgtctcgcaataatcatttacccgcttgtctaagttgtaacggaa

SEQ ID NO: 68 - RNA, 194 nt, Cowpox virus
  source 1..194: mol_type = genomic RNA
accatccaacggaaagtgtaaagacaacgaatacaaacgccatcatctatgttgtttatcgtgtcctccgggaacatacgcttccagattatgcgatagcaagactaacacaaacacacaatgtacgccgtgtgcgtcggacacctttacgtctcgcaataatcatttacccgcttgtctaagttgtaacggaa

SEQ ID NO: 69 - RNA, 164 nt, Vaccinia virus
  source 1..164: mol_type = genomic RNA
accatccgatggaaagtgtaaagacaacgaatacaaacgccataatttgtgtccgggaacatacgcttccgatagcaagactaacacacgatgtacgccgtgtggttcgggtaccttcacatctcgcaataatcatttacccgcttgtctaagttgtaacggaa

SEQ ID NO: 70 - RNA, 173 nt, Rabbitpox virus
  source 1..173: mol_type = genomic RNA
accatccgatggaaagtgtaaagacaacgaatacaaacgccataatttgtgtccgggaacatacgcttccagattatgcgatagcaagactaacacacgatgtacaccctgtggttcgggtaccttcacatctcgcaataatcatttacccgcttgtctaagttgtaacggaa

SEQ ID NO: 71 - RNA, 188 nt, Taterapox virus
  source 1..188: mol_type = genomic RNA
accatccaatggaaagtgtaaagacaacgaatacaaacgccataatctgtgttgtttatcgtgtcctccgggaacatacgcttccagattatgcgatatcaagactaacacacaatgtacgccgtgtggttcgggtaccttcacatctcacaataatcatttacccacttgtctaagttgtaacggaa

SEQ ID NO: 72 - RNA, 188 nt, Horsepox virus
  source 1..188: mol_type = genomic RNA
accatccaatggaaagtgtaaagacaacgaatacaaacgccataatttgtgttgtttatcgtgtcctccgggaacatacgcttccagattatgcgatagcaagactaacacacaatgtacgccgtgtggttcgggtaccttcacatctcgcaataatcatttacccgcttgtctaagttgtaacggaa

SEQ ID NO: 73 - RNA, 235 nt, Monkeypox virus
  source 1..235: mol_type = genomic RNA
cagcattgatttcattattacgtccgcagtcgttcaactgtatttcaagatctgagattctagattgtaatctctgtagcatttccacggcattcactcagttgtctttcaagatctgagattctagattggagtctgctaatctctgtaagatttcctcctccgctctcgatgcagtcggtcaacttattctctagttctctaatacgcgaacgcagtgcatcaacttcttgtg

SEQ ID NO: 74 - RNA, 235 nt, Monkeypox virus; identical to SEQ ID NO: 73
  source 1..235: mol_type = genomic RNA

SEQ ID NO: 75 - RNA, 235 nt, Monkeypox virus; identical to SEQ ID NO: 73
  source 1..235: mol_type = genomic RNA

SEQ ID NO: 76 - RNA, 235 nt, Monkeypox virus
  source 1..235: mol_type = genomic RNA
cagcattgatttcattattacgttcgcagtcgttcaactgtatttcaagatctgagattctagattgtaatctctgtagcatttccacggcattcactcagttgtctttcaagatctgagattctagattggagtctgctaatctctgtaagatttcctcctccgctctcgatgcagtcggtcaacttattctctagttctctaatacgcgaacgcagtgcatcaacttcttgtg

SEQ ID NO: 77 - RNA, 235 nt, Monkeypox virus; identical to SEQ ID NO: 76
  source 1..235: mol_type = genomic RNA

SEQ ID NO: 78 - RNA, 235 nt, Monkeypox virus; identical to SEQ ID NO: 76
  source 1..235: mol_type = genomic RNA

SEQ ID NO: 79 - RNA, 235 nt, Monkeypox virus; identical to SEQ ID NO: 76
  source 1..235: mol_type = genomic RNA

SEQ ID NO: 80 - RNA, 235 nt, Monkeypox virus; identical to SEQ ID NO: 76
  source 1..235: mol_type = genomic RNA

SEQ ID NO: 81 - RNA, 235 nt, Monkeypox virus; identical to SEQ ID NO: 76
  source 1..235: mol_type = genomic RNA

SEQ ID NO: 82 - RNA, 235 nt, Monkeypox virus; identical to SEQ ID NO: 76
  source 1..235: mol_type = genomic RNA

SEQ ID NO: 83 - RNA, 235 nt, Monkeypox virus; identical to SEQ ID NO: 76
  source 1..235: mol_type = genomic RNA

SEQ ID NO: 84 - RNA, 235 nt, Monkeypox virus; identical to SEQ ID NO: 76
  source 1..235: mol_type = genomic RNA

SEQ ID NO: 85 - RNA, 235 nt, Monkeypox virus; identical to SEQ ID NO: 76
  source 1..235: mol_type = genomic RNA

SEQ ID NO: 86 - RNA, 232 nt, Monkeypox virus
  source 1..232: mol_type = genomic RNA
gtacattgatttcattattacgtccgcagtcgttctgtatttcaagatctgagattctagattgtaatctctgtagcatttccacggcattcactcagttgtctttcaagatctgagattctagattggagtctgctaatctctgtaagatttcctcctccgctctcgatgcagtcggtcaacttattctctagttctctaatacgtgaacgcagtgcatcaacttcttgtg

SEQ ID NO: 87 - RNA, 262 nt, Variola virus
  source 1..262: mol_type = genomic RNA
cagcattgctttcattattacgtctgtagtcacttaactatctttcaagatttgaaattctatctctaagacgtctcatttctatctgtttcggtattggtcttattattatgtctacagtcgttcaactgtctttcaagatctaatattctagattggagtctactaatctctgtaagatttcctcctccgctctcaatgcagtcggtcaacttattctctagttcgctaatacgcgaacgcagtgcatcaacttcttgcg

SEQ ID NO: 88 - RNA, 262 nt, Variola virus; identical to SEQ ID NO: 87
  source 1..262: mol_type = genomic RNA

SEQ ID NO: 89 - RNA, 334 nt, Camelpox virus
  source 1..334: mol_type = genomic RNA
cagcattgctttcataattacgtctgcagtcactcaattgtctttcaagatctgagattctatctctaagacgtcgcatctctctctgtttcggcattggtctcattattacgtctacagtcgttcaactgtctttcaagatctgatattctagattcgagtctgctaatctctgtaacatttccacggcattcattcagttgtctttcaagatctgagattctagattggagtctgctaatctctgtaagatttcctcctccgctctcgatgcagtcggtcaacttattctctagttctctaatacgcgaacgcagtgcatcaacttcttgcg

SEQ ID NO: 90 - RNA, 334 nt, Camelpox virus
  source 1..334: mol_type = genomic RNA
cagcattgctttcataattacgtctgcagtcactcaactgtctttcaagatctgagattctatctctaagacgtcgcatctctctctgtttcggcattggtctcattattacgtctacagtcgttcaactgtctttcaagatctgatattctagattcgagtctgctaatctctgtaacatttccacggcattcattcagttgtctttcaagatctgagattctagattggagtctgctaatctctgtaagatttcctcctccgctctcgatgcagtcggtcaacttattctctagttctctaatacgcgaacgcagtgcatcaacttcttgcg

SEQ ID NO: 91 - RNA, 336 nt, Cowpox virus
  source 1..336: mol_type = genomic RNA
cagcattgctttcattattacgtctgcagtcactcaactgtctttcaagattcgtgattctatctctaagacgtcgcatctctctttctgtttcggcattggtctcattattacgtctacagtcgttcaactgtctttcaagatctgagattctagattggagtctgttaatctctgtaacatttccacggcattcactcagttgtcgttcaagatctgagattctagatcggagtctgctaatctctgtaagatttcctcctccactctcgatgcagtcggccaacttattctctagttctctaatacgcgaacgcagtgcatcaacttcttgcg

SEQ ID NO: 92 - RNA, 243 nt, Cowpox virus
  source 1..243: mol_type = genomic RNA
cggcattgttctcattattacgtctacagtcgttcagttgtctttcaagatctgtgattctatattggagtctgctaatctctgtagcatttccacggcattcacgcagttgtctttcaagatctgagattttagattggagtctgctaatctctgtgagatttcctcctccgctttccatgcagtcggccaacttattctccagttctctaatacgcgaacgcagtgcatcaacttcttgcg

SEQ ID NO: 93 - RNA, 334 nt, Vaccinia virus
  source 1..334: mol_type = genomic RNA
cagcattgctttcattattacgtctgcagtcactcaactgtctttcaatatctgagattctatctctaagacgtcgcatctctctctgtttcggcattggtttcattattacgtctacagtcgttcaactgtctttcaagatctgatattctagattggagtctgctaatctctgtagcattttcacggcattcactcagttgtctttcaagatctgagattttagattggagtctgctaatctctgtaagatttcctcctccgctctcgatgcagtcggtcaacttattctctagttctctaatacgcgaacgcagtgcatcaacttcttgcg

SEQ ID NO: 94 - RNA, 334 nt, Rabbitpox virus; identical to SEQ ID NO: 93
  source 1..334: mol_type = genomic RNA

SEQ ID NO: 95 - RNA, 332 nt, Taterapox virus
  source 1..332: mol_type = genomic RNA
cagcattggtctcattattacgtctacagtcgttcaactgtctttcaagatctgagattctatctctaagacgtcgcatctctctgtttcggcattggtctcattattacgtctacagtcgttcaactctctttcaagatctgatattctagattggagtctgctaatctctgtagcatttccacggcattcactcagttgtctttcaagatctgagattctagattggagtctgctaatctctgtaagatttcctcctccgctctcgatgcagtcggtcaacttattctctagttctctaatacgcgaacgcagtgcatcaacttcttgcg

SEQ ID NO: 96 - RNA, 334 nt, Horsepox virus
  source 1..334: mol_type = genomic RNA
cagcattgctttcattattacgtctgcagtcactcaactgtctttcaatatctgagattctatctctaagacgtcgcatctctctctgtttcggcattggtttcattattacgtctacagtcgttcaactgtctttcaagatctgatattctagattggagtctgctaatctctgtagcattttcacggcattcactcagttgtctttcaagatctgagattctagattggagtctgctaatctctgtaagatttcctcctccgctctcgatgcagtcggtcaacttattctctagttctctaatacgcgaacgcagtgcatcaacttcttgcg

SEQ ID NO: 97 - RNA, 228 nt, Severe acute respiratory syndrome-related coronavirus; identical to SEQ ID NO: 49
  source 1..228: mol_type = genomic RNA

SEQ ID NO: 98 - RNA, 228 nt, Severe acute respiratory syndrome-related coronavirus
  source 1..228: mol_type = genomic RNA
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcttgatcttctggtctaa

SEQ ID NO: 99 - RNA, 228 nt, Severe acute respiratory syndrome-related coronavirus; identical to SEQ ID NO: 49
  source 1..228: mol_type = genomic RNA

SEQ ID NO: 100 - RNA, 228 nt, Severe acute respiratory syndrome-related coronavirus; identical to SEQ ID NO: 49
  source 1..228: mol_type = genomic RNA

SEQ ID NO: 101 - RNA, 228 nt, Severe acute respiratory syndrome-related coronavirus
  source 1..228: mol_type = genomic RNA
atgtactcattcgtttcggaagagataggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 102 - RNA, 228 nt, Severe acute respiratory syndrome-related coronavirus; identical to SEQ ID NO: 101
  source 1..228: mol_type = genomic RNA

SEQ ID NO: 103 - RNA, 228 nt, Severe acute respiratory syndrome-related coronavirus; identical to SEQ ID NO: 101
  source 1..228: mol_type = genomic RNA

SEQ ID NO: 104 - RNA, 228 nt, Severe acute respiratory syndrome-related coronavirus; identical to SEQ ID NO: 101
  source 1..228: mol_type = genomic RNA

SEQ ID NO: 105 - RNA, 228 nt, Severe acute respiratory syndrome-related coronavirus; identical to SEQ ID NO: 101
  source 1..228: mol_type = genomic RNA

SEQ ID NO: 106 - RNA, 228 nt, Severe acute respiratory syndrome-related coronavirus; identical to SEQ ID NO: 49
  source 1..228: mol_type = genomic RNA

SEQ ID NO: 107 - RNA, 228 nt, Severe acute respiratory syndrome-related coronavirus
  source 1..228: mol_type = genomic RNA
atgtactcattcgtttcggaagagacaggtacgttaatagttaatagcgtacttcttttttttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 108 - RNA, 228 nt, Severe acute respiratory syndrome-related coronavirus; identical to SEQ ID NO: 49
  source 1..228: mol_type = genomic RNA

SEQ ID NO: 109 - RNA, 228 nt, Severe acute respiratory syndrome-related coronavirus; identical to SEQ ID NO: 49
  source 1..228: mol_type = genomic RNA

SEQ ID NO: 110 - RNA, 228 nt, Severe acute respiratory syndrome-related coronavirus; identical to SEQ ID NO: 49
  source 1..228: mol_type = genomic RNA

SEQ ID NO: 111 - RNA, 228 nt, Severe acute respiratory syndrome-related coronavirus; identical to SEQ ID NO: 49
  source 1..228: mol_type = genomic RNA